(19) United States
(12) Patent Application Publication
(10) Pub. No.: US 2011/0009607 A1
(43) Pub. Date: Jan. 13, 2011

(54) METHOD FOR PREPARING DNA FRAGMENT HAVING STICKY END

(76) Inventors: Makoto Komiyama, Tokyo (JP); Akinori Kuzuya, Tokyo (JP); Keita Tanaka, Tokyo (JP)

Correspondence Address: BIRCH STEWART KOLASCH & BIRCH, PO BOX 747, FALLS CHURCH, VA, 22040-0747, US

(21) Appl. No.: 12/921,930

(22) PCT Filed: Mar. 10, 2009
(86) PCT No.: PCT/JP2009/054989

§ 371 Date: Sep. 10, 2010

(30) Foreign Application Priority Data
Mar. 11, 2008 (JP) 2008-061678

(51) Int. Cl. C12P 19/34; C07H 21/04; C07C 205/06; C07D 239/54; C07D 473/28; C07H 19/09; C07H 19/19

(52) U.S. Cl. 536/24.33; 435/91.2; 536/23.1; 568/583; 544/309; 544/276; 536/28.54; 536/27.4

(57) ABSTRACT

The present invention provides a method for preparing a DNA fragment, in which a desired double-stranded DNA fragment having a sticky end is directly and easily obtained from an amplification product (an amplified fragment) after PCR without a restriction enzyme digestion. The method for preparing a DNA fragment having a sticky end of the present invention comprises: (i) a step of performing a PCR reaction using a template DNA and specific primers to obtain an amplified DNA fragment; and (ii) a step of performing a prescribed treatment on the amplified DNA fragment to dissociate a protecting group from the fragment. Herein, the above-mentioned specific primers are composed of a complementary DNA portion consisting of a nucleotide sequence complementarily binding to an amplification target region in a template DNA and a non-complementary DNA portion consisting of a nucleotide sequence that links to the 5′ end of the complementary DNA portion but does not complementarily bind to the amplification target sequence, and at least a base corresponding to the 3′ end in the nucleotide sequence of the non-complementary DNA portion is modified with a protecting group capable of terminating the progression of DNA replication catalyzed by a DNA polymerase.

### TECHNICAL FIELD

The present invention relates to a method for directly preparing a desired double-stranded DNA fragment having a sticky end (a protruding end) without a restriction enzyme digestion or the like. Moreover, the present invention also relates to a genetic recombination method using the DNA fragment obtained by the aforementioned method.

### BACKGROUND ART

Conventionally, in the field of molecular biology and biotechnology, a plasmid DNA is cleaved by restriction enzymes, and the cleavage portion used as a host DNA is allowed to bind to a desired gene fragment using a ligase, so as to construct a vector. In many cases, the aforementioned gene fragment is prepared from various materials (template DNAs) by PCR, and in order to bind it to a plasmid DNA (a host DNA), the blunt ends of the PCR amplification product are digested with restriction enzymes, so that they are converted to sticky ends (protruding ends) (Sambrook, J. et al., 2001, Molecular Cloning: A Laboratory Manual Edn.3. Cold Spring Harbor Laboratory Press, Cold Spring Harbor, N.Y.). By this technique, a desired vector can be obtained at a relatively high efficiency. However, this technique has the following two problems. The first problem is that, since the restriction site is limited to several palindromes, it may be often difficult to find out restriction enzymes suitable for cleaving DNA at a site of interest (or around such site of interest). The second problem is that a majority of available restriction enzymes recognize a DNA sequence consisting of 4 to 6 bases, and that, when a DNA with a large size is digested with restriction enzymes (wherein many identical recognition sites exist), many sites other than a desired site are also cleaved. As a result, if a host DNA is large in size, precise genetic recombination operations cannot be conducted.

As a solution to such problems, the present inventor has combined a Ce⁴⁺/EDTA complex (molecular scissors) with a pair of pseudo-complementary peptide nucleic acids (pcPNAs) (invasion in a double-stranded DNA and production of a locally single-stranded region), so as to produce an artificial restriction DNA cutter (ARCUT). If such pcPNAs are allowed to act on a double-stranded DNA, the pcPNAs partially dissociate the double-stranded DNA and then bind it to their target sequence (inversion in a double-stranded DNA). Herein, if the target sequence of the pcPNAs is shifted by several bases, a side opposite to the pcPNA-binding portion locally remains as a single strand during the formation of a complex. If Ce⁴⁺/EDTA is allowed to act on this complex, the Ce⁴⁺/EDTA cleaves a single-stranded DNA more selectively than a double-stranded DNA. Thus, the single-stranded portion produced by the pcPNAs is selectively cleaved. Since the cleavage site of each DNA strand is shifted in the horizontal direction, a sticky end is formed after completion of the cleavage. The sticky end formed by ARCUT does not directly bind to the ends of other fragments that have been separately prepared (e.g. a restriction fragment). However, with the use of a joint oligonucleotide that fills the space between these fragments, the fragments apparently form a complementary structure, and they may bind to each other (Yamamoto, Y. et al., Chem. Bio. Chem., vol. 7, pp. 673-677, 2006). However, the method using ARCUT has been problematic in that it has low binding efficiency (ligation efficiency) and in that it becomes difficult to carry out purification/separation steps.

### DISCLOSURE OF THE INVENTION

Thus, it is an object of the present invention to provide a method for preparing a DNA fragment, in which a desired double-stranded DNA fragment having a sticky end is directly and easily obtained from an amplification product (an amplified fragment) after PCR without a restriction enzyme digestion. Moreover, it is another object of the present invention to provide a genetic recombination method and the like using the DNA fragment obtained by the above-described preparation method.

The present inventor has conducted intensive studies directed towards achieving the aforementioned objects. As a result, the inventor has found that the aforementioned objects can be achieved with the use of a primer used for PCR, which has a protecting group having a specific function, thereby completing the present invention.

Specifically, the present invention is as follows:

(1) A primer used for PCR, which is composed of a complementary DNA portion consisting of a nucleotide sequence complementarily binding to an amplification target region in a template DNA and a non-complementary DNA portion consisting of a nucleotide sequence that links to the 5′ end of the complementary DNA portion but does not complementarily bind to the amplification target sequence, wherein at least a base corresponding to the 3′ end in the nucleotide sequence of the non-complementary DNA portion is modified with a protecting group capable of terminating the progression of DNA replication catalyzed by a DNA polymerase.

In the primer of the present invention, the protecting group can be dissociated from the modified base, for example, by a light irradiation treatment, an alkali treatment, an acid treatment, an oxidation treatment, a reduction treatment, a desilylation treatment, a heat treatment, an esterase treatment or a phosphatase treatment.

Herein, examples of the protecting group that can be dissociated from the modified base by the light irradiation treatment include a 2-(2-nitrophenyl)propyl group, a 2-(2-nitrophenyl)propyloxymethyl group, a 1-(2-nitrophenyl)ethyl group and a 6-nitropiperonyloxymethyl group. Examples of the protecting group that can be dissociated from the modified base by the alkali treatment include an isobutyryl group, a benzoyl group and an acetoxymethyl group. Examples of the protecting group that can be dissociated from the modified base by the acid treatment include a trityl group and a methoxy derivative thereof. Examples of the protecting group that can be dissociated from the modified base by the oxidation treatment include an allyloxymethyl group, a dimethoxybenzyloxymethyl group and a trimethoxybenzyloxymethyl group. Examples of the protecting group that can be dissociated from the modified base by the reduction treatment include a benzyloxymethyl group, and a benzyloxymethyl group substituted with any given substituent. Examples of the protecting group that can be dissociated from the modified base by the desilylation treatment include a t-butyldimethoxysilyloxymethyl group and a t-butyldiphenylsilyloxymethyl group. An example of the protecting group that can be dissociated from the modified base by the heat treatment is an isocyanate group. An example of the protecting group that can be dissociated from the modified base by the esterase treatment is an acetoxymethyl group. An example of the protecting group that can be dissociated from the modified base by the phosphatase treatment is a methyl phosphate group.

Moreover, in the primer of the present invention, examples of at least a base corresponding to the 3′ end in the nucleotide sequence of the non-complementary DNA portion include thymine and guanine. Furthermore, in the primer of the present invention, the non-complementary DNA portion is, for example, 1 to 100 bases in length.

(2) A method for preparing a DNA fragment having a sticky end, which comprises: (i) a step of performing a PCR reaction using a template DNA and the primer according to any one of claims 1 to 13 to obtain an amplified DNA fragment; and (ii) a step of performing a prescribed treatment on the amplified DNA fragment to dissociate a protecting group from the fragment.

In the preparation method of the present invention, the prescribed treatment for dissociating the protecting group from the modified base is, for example, a light irradiation treatment, an alkali treatment, an acid treatment, an oxidation treatment, a reduction treatment, a desilylation treatment, a heat treatment, an esterase treatment or a phosphatase treatment.

In the preparation method of the present invention, the sticky end is, for example, a sticky end having the same nucleotide sequence as that of a sticky end obtained by a restriction enzyme digestion, or a sticky end having a nucleotide sequence different from that of a sticky end obtained by a restriction enzyme digestion.

(3) A genetic recombination method, which comprises a step of binding the DNA fragment obtained by the preparation method according to (2) above to a host DNA.

In the genetic recombination method of the present invention, the binding operation of the above-described DNA fragment to the host DNA can be carried out, for example, without using a DNA ligase.

(4) A substituent-introducing agent comprising a compound represented by the following formula (I′):

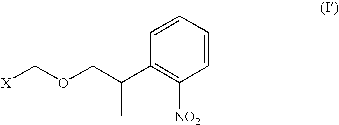

[wherein, in the formula (I′), X represents Cl, I, Br, p-toluenesulfonic acid ester or sulfuric acid ester].

(5) A biomolecule having a 2-(2-nitrophenyl)propyloxymethyl group.

An example of the biomolecule of the present invention is a biomolecule represented by the following formula (I):

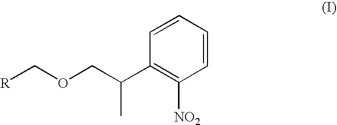

[wherein, in the formula (I), R represents a biomolecule].

Herein, an example of the biomolecule is a base, and specific examples include thymine and guanine. When the biomolecules are thymine and guanine, examples of the biomolecule of the present invention include those represented by the following formulae (II) and (III):

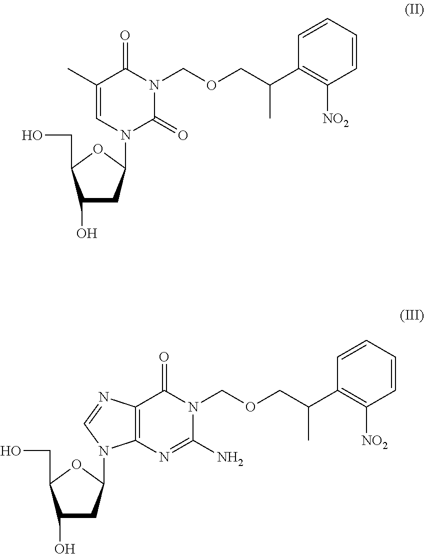

### BRIEF DESCRIPTION OF THE DRAWINGS

FIG. 1 is a schematic view of LACE-PCR (Light-assisted-cohesive-ending PCR) using caged T^{NPP}, which is one example of the present invention.

FIG. 2 is a photograph of an electrophoresis (20% denaturing PAGE) showing the results of an elongation reaction catalyzed by a DNA polymerase. Lane 1 shows only C2 (control), lane 2 shows C1+C2+Ex Taq (without UV irradiation), and lane 3 shows C1+C2+Ex Taq (with UV irradiation). The reaction conditions are [C1]=[C2]=4 μM, [dNTPs]=100 μM, [Ex Taq]=0.2 U/μl, 37° C., and 30 minutes.

FIG. 3 is a schematic view of the construction of a recombinant vector by the LACE-PCR method.

FIG. 4 is a view showing the structure of an insert DNA fragment prepared by the LACE-PCR method.

FIG. 5 is a photograph showing an electrophoresis of an amplification product by the LACE-PCR method.

FIG. 6 is a photograph of an electrophoresis showing the results of colony PCR.

FIG. 7 is a view showing the results of the analyses of the nucleotide sequences of recombinant vectors.

FIG. 8 is a graph showing a change over time in the deprotection of a protecting group (NPP) by UV irradiation.

FIG. 9 is a view showing the structure of an insert DNA fragment prepared by the LACE-PCR method.

FIG. 10 is a photograph showing an electrophoresis of an amplification product by the LACE-PCR method, which is carried out before and after UV irradiation. Lane 1 shows before UV irradiation, and lane 2 shows after UV irradiation.

FIG. 11 is a photograph of an electrophoresis showing the results of colony PCR.

FIG. 12 is a view showing the results of the analyses of the nucleotide sequences of recombinant vectors. FIG. 12(a) shows a recombinant vector, to which an Eco RI site has bound. FIG. 12(b) shows a recombinant vector, to which a Hind III site has bound.

FIG. 13 is a view showing target cleavage sites to be cleaved by ARCUT and the nucleotide sequences of pcPNAs. FIG. 13(a) shows a nucleotide sequence around a target site to be cleaved by ARCUT. FIG. 13(b) shows a structure around the target site that is cleaved by ARCUT.

FIG. 14(a) is a view showing the structure of a target fragment (vector) cleaved by ARCUT, and FIG. 14(b) is a view showing the structure of an insert DNA fragment (insert) prepared by the LACE-PCR method.

FIG. 15 is a view showing a restriction site on a pBR322 vector.

FIG. 16(a) is a view showing the structure of a target fragment (vector) cleaved by ARCUT, and FIG. 16(b) is a view showing the structure of an insert DNA fragment (insert) prepared by the LACE-PCR method.

FIG. 17 is a photograph showing an agarose gel electrophoresis of the cleavage fragment of a pBR322 vector cleaved by EcoRI or ARCUT. Lane 1 shows a fragment cleaved by only EcoRI, lane 2 shows a fragment cleaved by EcoRI and then by ARCUT, and lane M shows a 1 kbp ladder marker.

FIG. 18 is a photograph showing an agarose gel electrophoresis of individual fragments used or obtained in the present Examples. Lane 1 shows an ARCUT cleavage fragment (2530 bp), lane 2 shows an amplification product obtained by the LACE-PCR method (with UV irradiation), lane 3 shows only a recombinant vector, and lane 4 shows a recombinant vector cleaved by EcoPI.

FIG. 19 is a view showing the results of the analyses of the nucleotide sequences of recombinant vectors. FIG. 19(a) shows a recombinant vector, to which an ARCUT site has bound. FIG. 19(b) shows a recombinant vector, to which an Eco RI site has bound.

FIG. 20 is a photograph showing GFP luminescence in a transformant of BL21-Gold (DE3).

FIG. 21 is a schematic view showing the construction of a vector comprising a DNA encoding a GFP-BFP fusion protein. The left view shows a pQBI T7-GFP vector comprising a GFP gene, and the right view shows a pQBI 67 vector comprising a BFP gene.

FIG. 22 is a photograph showing an agarose gel electrophoresis of a product obtained by amplifying a DNA fragment comprising a BFP gene by LACE-PCR.

FIG. 23 is a photograph showing the luminescence of a GFP-BFP fusion protein or the like in a transformant of BL21-Gold (DE3).

FIG. 24 is a photograph showing an agarose gel electrophoresis of a recombinant vector recovered from a transformant of BL21-Gold (DE3).

FIG. 25 is a view showing the fluorescence spectrum of a GFP-BFP fusion protein.

FIG. 26 is a photograph showing the results of SDS-PAGE analysis of a protein expressing in a transformant of BL21-Gold (DE3).

### BEST MODE FOR CARRYING OUT THE INVENTION

The present invention will be described in detail below. The following descriptions are not intended to limit the scope of the present invention. Other than the following examples, the present invention may be modified and may be carried out, as appropriate, within a range that does not impair the intention of the present invention. The present specification includes all of the contents as disclosed in the specification of Japanese Patent Application No. 2008-061678 (filed on Mar. 11, 2008), which is a priority document of the present application. Moreover, all publications cited in the present specification, which include prior art documents and patent documents such as laid-open application publications and patent publications, are incorporated herein by reference in their entirety.

### 1. Summary of the Present Invention

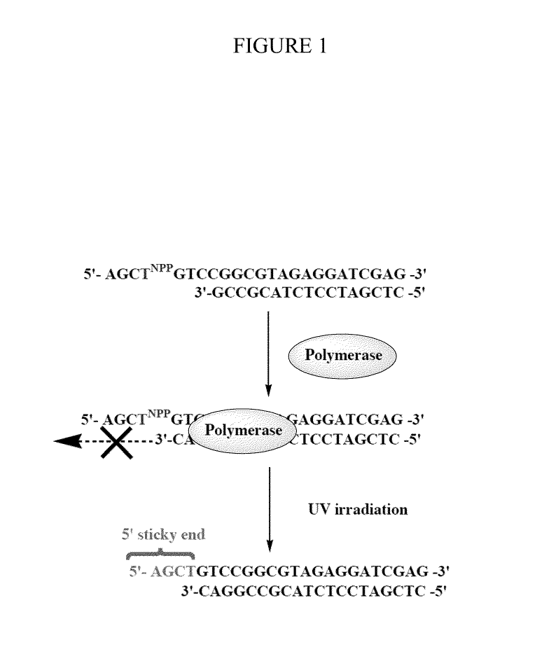

As shown in FIG. 1, if a modification has been made to inhibit base-pair formation between dNTPs and a template DNA (T^{NPP}; a thymidine residue modified with (protected by) a 2-(2-nitrophenyl)propyl group), an elongation reaction catalyzed by a DNA polymerase is inhibited, and as a result, a DNA fragment having a 5′ protruding end can be easily produced. The thus modified (protected) DNA fragment cannot be directly applied to genetic recombination. However, since a 2-(2-nitrophenyl)propyl group (NPP group) serving as a protecting group for T^{NPP} can be dissociated by light irradiation, the obtained DNA fragment can be easily used for genetic recombination technology and the like. The present inventor has found that this technique is applied in combination with a PCR technology, so as to create a method for arbitrarily designing a 5′ protruding end (sticky end) on an insert side that can be used in genetic recombination and the like, thereby completing the present invention.

Hence, the present inventor has performed the aforementioned modification on some bases to synthesize various PCR primers (see a C1 primer shown in Scheme 1 of Example 1 as described later, for example). Thereafter, the inventor has inserted the obtained PCR amplification product acting as an insert fragment into a vector without a restriction enzyme digestion, so as to construct a recombinant vector. By the way, it has been known that, in the case of a DNA cleavage fragment cleaved by ARCUT (an artificial restriction DNA cutter; see Yamamoto, Y. et al., Chem. Bio. Chem., vol. 7, pp. 673-677, 2006), due to the properties thereof, the nucleotide sequence of the cleavage portion must not be a sequence as in the case of using natural restriction enzymes. Thus, a vector is cleaved by ARCUT, and a DNA fragment serving as an insert is then prepared by the method of the present invention, such that it is matched with the nucleotide sequence of the aforementioned cleavage portion. Thereafter, the DNA fragment was attempted to be inserted into the vector. Conventionally, in genetic recombination using ARCUT, when an insert fragment has been ligated to a vector, it has been necessary to use an additional fragment called “Oligojoint” to fill the space between them. However, if the insert fragment prepared by the method of the present invention is used, such insert fragment could be extremely easily inserted into a vector without using Oligojoint. Several methods for producing a DNA fragment having a desired protruding end (sticky end) have been reported, so far. However, all of such methods have required a special enzyme digestion after completion of PCR (Aslanidis, C. et al., (1990) Ligation-independent cloning of PCR products (LIC-PCR). Nucleic Acids Res., 18, 6069-6074.; Bitinaite, J. et al., (2007) USER™ friendly DNA engineering and cloning method by uracil excision. Nucleic Acids Res., 35, 1992-2002.; Xin, W. et al., (2003) Construction of linear functional expression elements with DNA and RNA hybrid primers: a flexible method for proteomics., 25, 273-277.; Zhu, B. et al., (2007) In-Fusion™ assembly: seamless engineering of multidomain fusion proteins, modular vectors, and mutations. BioTechniques, 43, 354-359). According to the method of the present invention, a DNA fragment having a desired protruding end can be produced using substantially only a DNA polymerase as an enzyme. Since this method has a few limits (a few conditions), it is considered to be used for various applications.

### 2. Method for Preparing DNA Fragment Having Sticky End

The method for preparing a DNA fragment having a sticky end according to the present invention comprises the following steps (i) and (ii):

(i) a step of performing a PCR reaction using a template DNA and a prescribed primer used for PCR to obtain an amplified DNA fragment; and
(ii) a step of performing a prescribed treatment on the amplified DNA fragment to dissociate a protecting group from the fragment.

Herein, the prescribed PCR primer used in the aforementioned step (i) is a primer, which is composed of a complementary DNA portion consisting of a nucleotide sequence complementarily binding to an amplification target region in a template DNA that is a part of the template DNA, and a non-complementary DNA portion consisting of a nucleotide sequence that links to the 5′ end of the complementary DNA portion but does not complementarily bind to the amplification target sequence; the primer being characterized in that at least a base corresponding to the 3′ end in the nucleotide sequence of the non-complementary DNA portion is modified with a protecting group capable of terminating the progression of DNA replication catalyzed by a DNA polymerase. Herein, the “non-complementary DNA portion” in the concerned primer may be a portion consisting of a nucleotide sequence that is not complementary to the PCR amplification target region in the template DNA. Accordingly, the “non-complementary DNA portion” may also be a portion consisting of a nucleotide sequence complementary to a region other than the amplification target region in the template DNA (for example, a nucleotide sequence region with any given length adjacent to the 5′ or 3′ side of the amplification target region). Thus, the non-complementary DNA portion is not limited.

In the concerned primer, the non-complementary DNA portion is a portion that serves as a sticky end of the finally obtained DNA fragment (a single-stranded DNA portion). Herein, the base length of the non-complementary DNA portion is not particularly limited. It is, for example, 1 to 100 bases, and preferably 4 to 20 bases. In particular, according to the preparation method of the present invention, the base length of the sticky end of the obtained DNA fragment can be easily set at 5 bases or more, which cannot be generally realized by the sticky end after a restriction enzyme digestion. Accordingly, the sticky end of the obtained DNA fragment may consist of a nucleotide sequence that is either identical to or different from that of a sticky end obtained by a known restriction enzyme digestion. Thus, the sticky end is not limited.

The concerned primer has a protecting group capable of terminating the progression of DNA replication catalyzed by a DNA polymerase, which modifies the aforementioned prescribed base (at least a base corresponding to the 3′ end in the nucleotide sequence of the non-complementary DNA portion). The type of the base is not limited. Thymine and guanine are preferable. In general, in PCR operations, a complementary strand is synthesized also for the non-complementary DNA portion in the primer during the process of DNA synthesis catalyzed by a DNA polymerase, and DNA replication progresses. In the present invention, however, since the base located between the complementary DNA portion and the non-complementary DNA portion in the primer is modified with the aforementioned protecting group, such complementary strand is not synthesized for the non-complementary DNA portion during the DNA synthesis process in PCR. Accordingly, the obtained DNA fragment has a sticky end that corresponds to the non-complementary DNA portion.

Herein, the aforementioned protecting group is not limited, as long as it can be dissociated from the DNA fragment obtained after PCR by the prescribed treatment. Preferred examples include protecting groups described in the cited document, Green et al. (Protective Groups in Organic Synthesis, John Wiley and Sons, 2^{nd} edition, 1991). In the present invention, protecting groups that can be dissociated from the modified base by a light irradiation treatment, an alkali treatment, an acid treatment, an oxidation treatment, a reduction treatment, a desilylation treatment, a heat treatment, an esterase treatment or a phosphatase treatment, are preferable. Among these treatments, a light irradiation treatment (particularly preferably, a UV irradiation treatment), an alkali treatment, an oxidation treatment, a reduction treatment, a desilylation treatment and the like are more preferable. Accordingly, various types of treatments as listed above can be exemplified as prescribed treatments that are carried out in the above step (ii).

In the case of the above-described light irradiation treatment (particularly preferably, a UV irradiation treatment), for example, light is applied preferably for 5 to 60 minutes, and more preferably for 15 to 30 minutes, although the conditions depend on the type of a protecting group treated.

Hereinafter, protecting groups that can be dissociated by such light irradiation treatment will be exemplified. The groups enclosed with broken lines correspond to the concerned protecting groups.

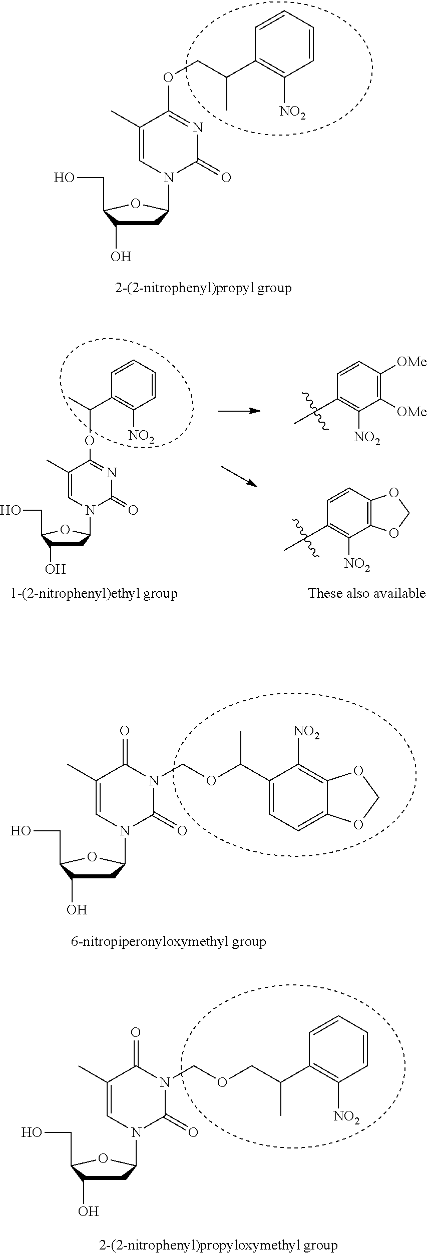

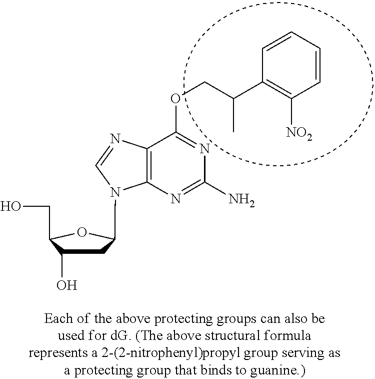

In the case of the above-described alkali treatment, concentrated ammonia water, a mixed solution of concentrated ammonia water and methylamine and the like are preferably used as reagents, for example. Concentrated ammonia water is more preferable. The alkali treatment is carried out, preferably at room temperature for 24 hours or at 55° C. for 8 hours, and more preferably at room temperature for 24 hours, although the conditions depend on the type of a protecting group treated.

Hereinafter, protecting groups that can be dissociated by such alkali treatment will be exemplified. The groups enclosed with broken lines correspond to the concerned protecting groups.

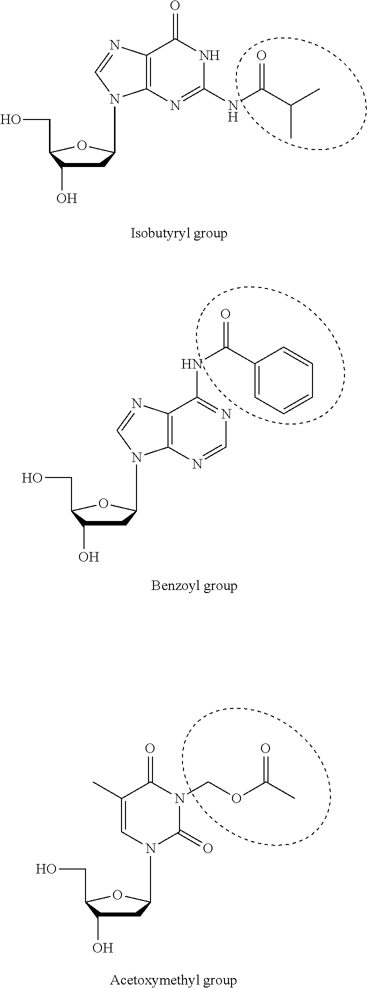

In the case of the above-described acid treatment, a 2%-50% trifluoroacetic acid aqueous solution, 10%-80% acetic acid, diluted hydrochloric acid and the like are preferably used as reagents, for example. A 2% trifluoroacetic acid aqueous solution and 80% acetic acid are more preferable. The acid treatment is carried out, preferably at room temperature for 10 minutes to overnight, and more preferably at room temperature for 1 hour, although the conditions depend on the type of a protecting group treated.

Preferred examples of a protecting group that can be dissociated by such acid treatment include a trityl group and a methoxy derivative thereof.

In the case of the above-described oxidation treatment, 2,3-dichloro-5,6-dicyano-p-benzoquinone, iodine and the like are preferably used as reagents, for example. 2,3-Dichloro-5,6-dicyano-p-benzoquinone is more preferable. The oxidation treatment is carried out, for example, by adding 2,3-dichloro-5,6-dicyano-p-benzoquinone to the sample in an aqueous solution, and then stirring it at room temperature, preferably for one hour to several days, and more preferably for 10 hours, although the conditions depend on the type of a protecting group treated.

Preferred examples of a protecting group that can be dissociated by such oxidation treatment include an allyloxymethyl group, a dimethoxybenzyloxymethyl group and a trimethoxybenzyloxymethyl group.

In the case of the above-described reduction treatment, with regard to reagents used, palladium-carbon, a Raney catalyst, an Adam's catalyst, a Lindlar's catalyst and the like are preferably used as catalysts combined with hydrogen gas, for example. Of these, palladium-carbon is more preferable. In addition, a single use of sodium borohydride or sodium cyanoborohydride is also preferable. The reduction treatment is carried out by adding a catalyst and hydrogen gas to a reaction system, and then reacting them at room temperature, preferably for overnight to several days, and more preferably overnight, although the conditions depend on the type of a protecting group treated.

Preferred examples of a protecting group that can be dissociated by such reduction treatment include a benzyloxymethyl group, and a benzyloxymethyl group which is substituted with any given substituent. Examples of such any given substituent include various types of known substituents such as those included in an alkyl group, an alkenyl group, an aryl group, an aralkyl group, a cycloalkyl group, an acyl group and a heterocyclic group. In the case of the above-described desilylation treatment, a 1 M tetrabutylammonium fluoride (THF) solution, triethylamine trihydrofluoride and the like are preferably used as reagents, for example. A 1 M tetrabutylammonium fluoride (THF) solution is more preferable. The desilylation treatment is carried out by adding a 1 M tetrabutylammonium fluoride (THF) solution to a sample solution, and then reacting them at room temperature, preferably for overnight to several days, and more preferably overnight.

Protecting groups that can be dissociated by such desilylation treatment will be exemplified. The groups enclosed with broken lines correspond to the concerned protecting groups.

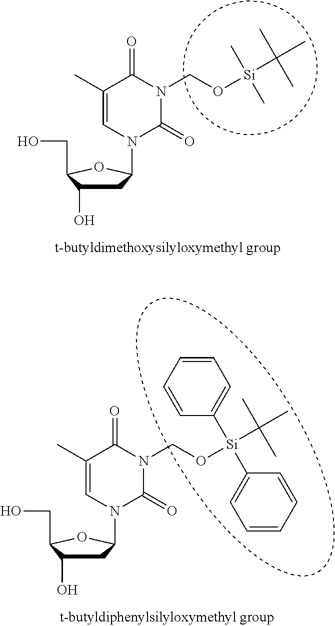

The above-described heat treatment is carried out, for example, by heating the reaction product comprising a protecting group, preferably at a temperature of 60° C. to 90° C. for 10 to 360 minutes, and more preferably at 60° C. for 10 minutes, although the conditions depend on the type of the protecting group treated.

A preferred example of protecting groups that can be dissociated by such heat treatment is an isocyanate group (see JP Patent Publication (Kokai) No. 2006-248931 A).

The above-described esterase treatment is carried out, for example, by reacting the reaction product comprising a protecting group with pig liver esterase, preferably at 37° C. for 1 hour or overnight, and more preferably at 37° C. for 1 hour, although the conditions depend on the type of the protecting group treated.

A protecting group that can be dissociated by such esterase treatment will be exemplified below. The group enclosed with broken line corresponds to the concerned protecting group.

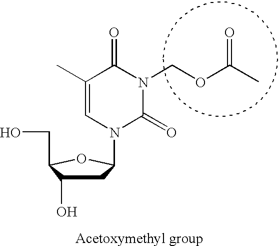

The above-described phosphatase treatment is carried out, for example, by reacting the reaction product comprising a protecting group with alkaline phosphatase, preferably at 37° C. for 1 hour or overnight, and more preferably at 37° C. for 1 hour, although the conditions depend on the type of the protecting group treated.

A protecting group that can be dissociated by such phosphatase treatment will be exemplified below. The group enclosed with broken line corresponds to the concerned protecting group.

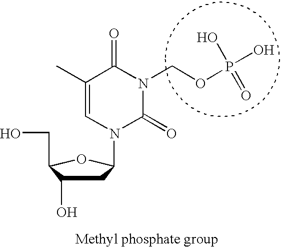

In the present invention, a known method for preparing a primer used for PCR having the aforementioned various types of protecting groups may be applied, and the preparation method is not limited. For example, the protection of a desired base by the aforementioned various types of protecting groups can be carried out: by activating with a tosyl compound, thymidine whose 5′ and 3′ hydroxyl groups have been protected by acetyl groups, and then allowing the alcohol form of a protecting group to act thereon; or by allowing a protecting group activated as a chloromethyl form to act on thymidine whose 5′ and 3′ hydroxyl groups have been protected by acetyl groups.

In the step (i) above, PCR conditions other than the above-described conditions, such as a reaction solution (a template concentration, a primer concentration, the type and concentration of a buffer, the type of a DNA polymerase, the amount of the enzyme, etc.) and reaction conditions (the reaction time and temperature of each of heat denaturation, annealing and elongation, the total cycle number, etc.), can be determined, as appropriate, based on known findings and common technical knowledge.

In the step (ii) above as well, the prescribed treatment for deprotection can be carried out by a known method, under known conditions, as well as the above-described conditions.

The preparation method of the present invention may also comprise other any given steps, as well as the above-described steps (i) and (ii). Such other any given steps are not particularly limited.

Moreover, the present invention can also provide a substituent-introducing agent for introducing a substituent serving as the above-described protecting group (specifically, a substituent-introducing agent comprising a compound that may act as such substituent), and it can further provide a biomolecule having such substituent (specifically, a protecting biomolecule).

Specifically, a preferred embodiment of the substituent-introducing agent of the present invention is an agent comprising a compound represented by the following formula (I′):

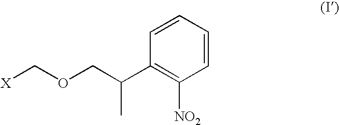

Herein, in the formula (I′), X is not limited, and it preferably represents Cl, I, Br, p-toluenesulfonic acid ester or sulfuric acid ester. Of these, a compound wherein, in the following formula, X is Cl, is more preferable (that is, 2-[(2-nitrophenyl)-chloromethoxy]-propane (NPPOM-Cl)):

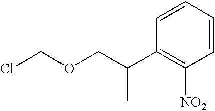

Herein, NPPOM-Cl can be synthesized, for example, according to the method of Example 3 as described later (see scheme 2). Moreover, the formation of a substituent using NPPOM-Cl can also be carried out according to the same above scheme. Furthermore, the synthesis of a compound wherein, in the formula (I′), X is a substance other than Cl, and the formation of a substituent, can also be carried out by referring to the case of NPPOM-Cl, based on common technical knowledge and the like. The substituent formed with the compound represented by the above formula (I′) (the substituent derived from the compound) is represented, for example, by a formula as shown below (specifically, a 2-(2-nitrophenyl)propyloxymethyl group).

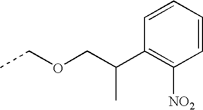

Further, the biomolecule of the present invention includes a biomolecule having a 2-(2-nitrophenyl)propyloxymethyl group (the above formula (I′)) (specifically, a biomolecule substituted with the aforementioned group). Specifically, there can be preferably used a biomolecule represented by the following formula (I):

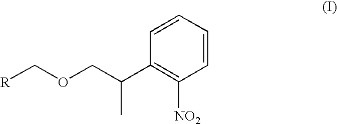

Herein, in the formula (I), R represents any given biomolecule. The Biomolecule® may be either a low-molecular-weight compound (a base, an amino acid, etc.) or a polymer (DNA, RNA, a protein, etc.). It may also be an artificially prepared biocompatible molecule (PNA, etc.). For example, a base is preferable. The type of such base is not particularly limited. Preferred examples of such base include thymine and guanine. When the Biomolecule® is thymine or guanine, preferred examples of the biomolecule of the present invention include a biomolecule represented by a formula (II) as shown below (specifically, a biomolecule whose thymine has been substituted with (protected by) a 2-(2-nitrophenyl)propyloxymethyl group):

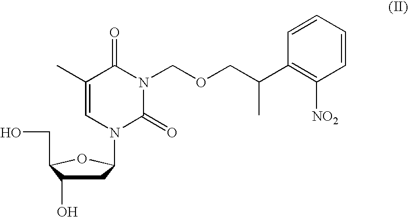

and a biomolecule represented by a formula (III) as shown below (specifically, a biomolecule whose guanine has been substituted with (protected by) a 2-(2-nitrophenyl)propyloxymethyl group):

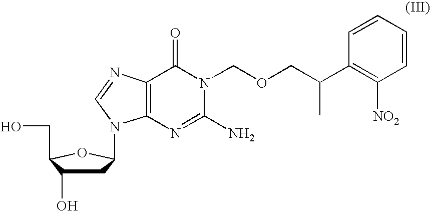

### 3. Genetic Recombination Method

The genetic recombination method of the present invention comprises a step of binding a DNA fragment obtained by the aforementioned preparation method of the present invention to a host DNA.

Herein, the type of a host DNA is not particularly limited. Any given DNAs, such as various types of vectors (an expression vector, a viral vector, a phage vector, etc.) or the genomic DNAs of various types of organisms, can be used.

As described above, the DNA fragment obtained by the preparation method of the present invention may desirably have a sticky end with a long base length, which cannot be obtained by a restriction enzyme digestion. In this case, the DNA fragment binds to a host DNA via their sticky ends having a long base length. Thus, although a DNA ligase such as a ligase is not particularly used, it becomes possible to bind the DNA fragment to the host DNA with high specificity and with high binding efficiency, only by mixing them. Therefore, the present invention can also provide a genetic recombination method that is excellent in terms of simplicity, economic efficiency and practical utility.

The genetic recombination method of the present invention may also comprise other any given steps, as well as the above-described binding step. Such other any given steps are not particularly limited.

The genetic recombination method of the present invention can be preferably used for the genetic modification of a retrovirus, an adenovirus and the like, which are used for gene therapy or in association with iPS cells. Since such adenovirus and the like have a large genomic DNA, excessive effort and time have been required for the genetic modification of such virus by the conventional method. According to the genetic recombination method of the present invention, the entire genome is divided into portions with a suitable size, and each portion is then amplified by PCR, separately. Thereafter, the amplification products are then ligated to one another via the sticky ends produced by the present invention, so as to easily carry out genetic recombination.

Moreover, the present invention can also provide a genetic recombination kit comprising the DNA fragment obtained by the above-described preparation method of the present invention. The kit may comprise other any given constitutional elements. For example, the kit may comprise various types of vectors such as an expression vector, various types of buffers, sterilized water, a preservative solution, an antiseptic, various types of reaction vessels, manual (instructions) and the like.

Hereinafter, the present invention will be more specifically described in the following examples. However, these examples are not intended to limit the scope of the present invention.

### Example 1

### Development of LACE-PCR (Light-Assisted-Cohesive-Ending PCR) Method and Application Thereof to ARCUT

### 1 Inhibition of DNA Polymerase Elongation Reaction by T^{NPP}

As shown in FIG. 1, in order to confirm the inhibition of an elongation reaction catalyzed by a DNA polymerase (hereinafter, in the present example, simply referred to as a polymerase), a polymerase elongation reaction was examined using C1 and C2 shown in the following Scheme 1. The nucleotide sequences of C1 to C6 shown in the Scheme 1 are shown in SEQ ID NOS: 1 to 6, respectively.

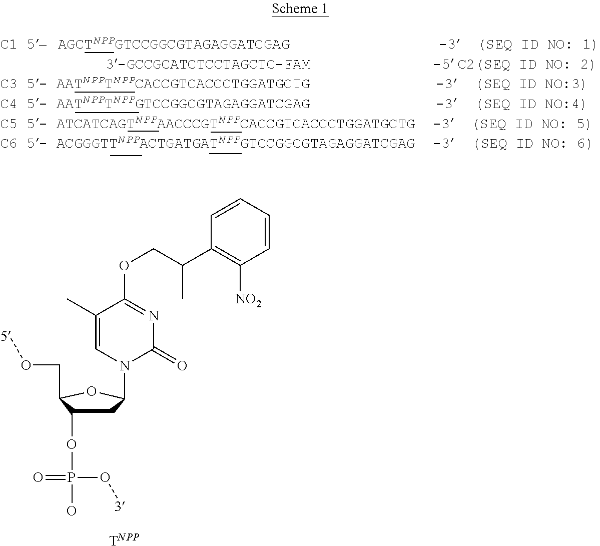

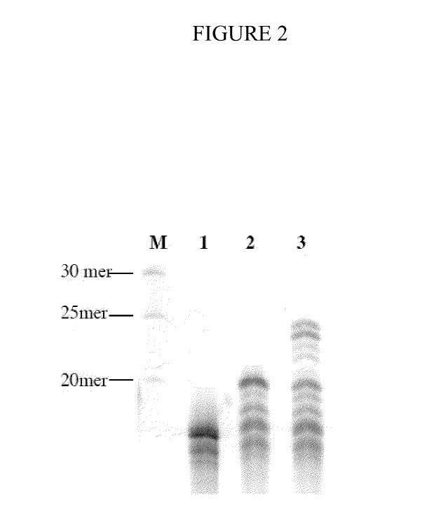

Herein, C1 acts as a template (a template DNA) of C2 used as a PCR primer, and has only one T^{NPP}. The method for synthesizing a primer having such T^{NPP} is as described above. The 5′ end of C2 was labeled with FAM. Various types of polymerases were used. Among them, when Ex Taq was used, the clearest results were obtained. The results of the reaction using such Ex Taq are shown in FIG. 2. In FIG. 2, C1, which had been irradiated with light (UV irradiation) before initiation of the reaction, was used in lane 3. When compared with lane 1, it was found that the elongation reaction in lane 3 progressed to a portion of 24 mer. However, the elongation reaction in lane 2 did not progressed when compared with lane 3, and it could be confirmed that the elongation terminated at a portion of interest (20 mer). With regard to the inhibition of the elongation reaction by C4 and C6 as well, it was confirmed that the elongation reaction terminated at a portion of interest (data not shown). These results yield two conclusions. The first conclusion is that the elongation reaction catalyzed by a polymerase can be terminated at a T^{NPP} of interest. The second conclusion is that the caged protection of T^{NPP} (specifically, protection from the DNA polymerase elongation reaction by an NPP group (inhibition of the elongation reaction)) is efficiently deprotected by UV irradiation. It was also found that, in order to terminate the elongation reaction, it is sufficient to contain only one T^{NPP}.

It has previously been known that the elongation reaction catalyzed by a polymerase is suppressed by a TT dimer. It has been reported that, utilizing such phenomenon, the elongation reaction is inhibited by a template in which two modified bases are aligned, and that the elongation reaction is then promoted by dissociating the one modified base by light irradiation (Tang, X. J. et al., (2005) Photoregulation of DNA polymerase I (Klenow) with caged fluorescent oligodeoxynucleotides. Bioorg. Med. Chem. Leu., 15, 5303-5306). Thus, the present inventor considered that the inhibition efficiency would be enhanced by introducing two T^{NPP} residues, and introduced such two T^{NPP} residues into the C3 primer.

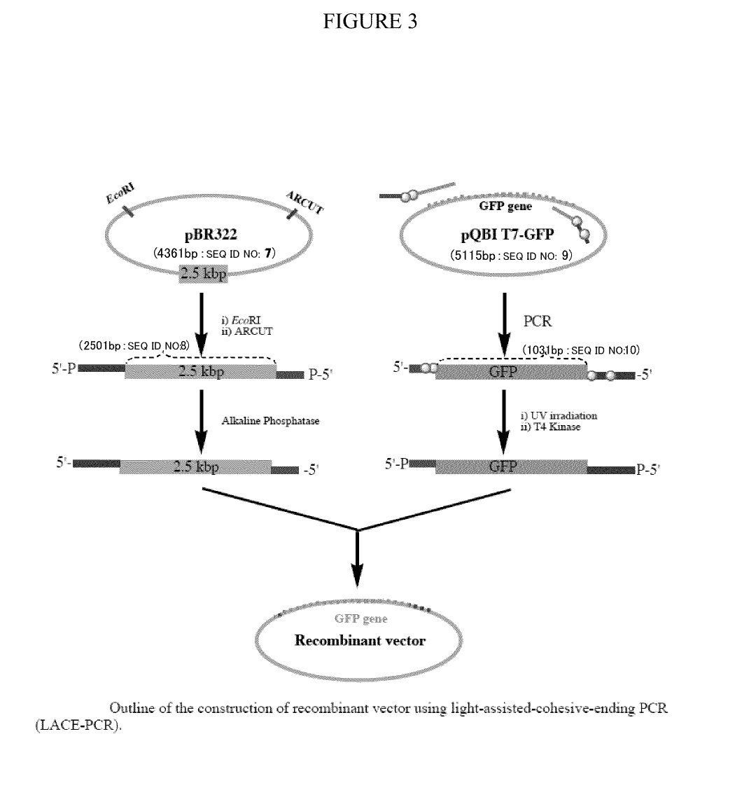

According to the method of the present invention, it became possible to produce a DNA fragment having a desired 5′ protruding end. Thus, various insert DNA fragments (inserts) were actually prepared according to PCR using desired synthetic primers, and the thus prepared inserts were each inserted into a vector according to a common method. In the present example, a summary of a method using a vector cleaved by restriction enzyme (EcoRI) or ARCUT is shown in FIG. 3. It is to be noted that, for ARCUT (an artificial restriction DNA cutter), “Yamamoto, Y. et al., Chem. Bio. Chem., vol. 7, pp. 673-677, 2006” can be referred.

2. Insertion into Single Cleavage Site by EcoRI

### 2-1. Designing and Synthesis of EcoRI LACE Primer

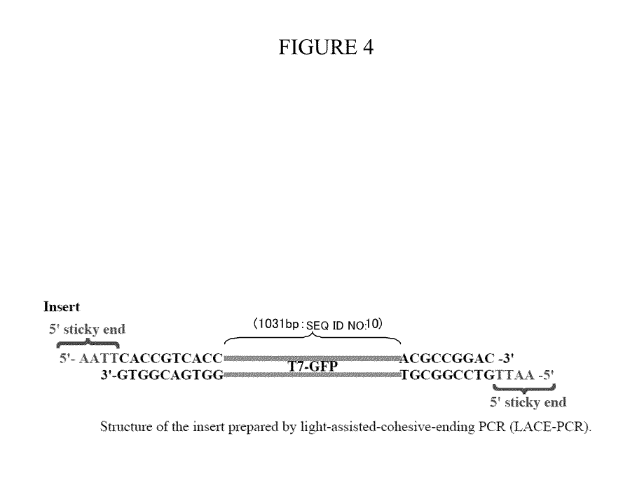

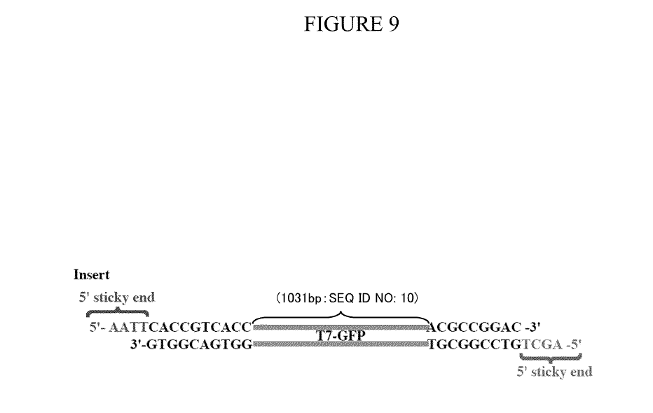

In order to cleave a vector (pUC18) by EcoRI and then insert the prepared insert into the cleaved portion, PCR primers C3 and C4, as shown in the Scheme 1, were synthesized. The primers C3 and C4 were designed, so that they were able to amplify a nucleotide sequence region (121-1170 bp) comprising both the GFP gene of pQBI T7-GFP (Qbiogene; 5115 bp (SEQ ID NO: 9)) and a T7 promoter sequence, and so that the insert obtained as an amplification product had a 5′ protruding end complementary to the EcoRI cleavage fragment (FIG. 4). In the sequence of the insert shown in FIG. 4, the nucleotide sequence portion indicated as T7-GFP (comprising the GFP gene and the T7 promoter sequence; total 1031 bp; consisting of bases 131-1161 in the nucleotide sequence shown in SEQ ID NO: 9) is shown in SEQ ID NO: 10 (the same applies to FIGS. 9, 14b and 16b). As stated above, in order to enhance inhibition efficiency, two T^{NPP} residues were introduced into each primer. Hereinafter, the primer used in LACE-PCR was referred to as a LACE primer.

2-2. Insertion of LACE-PCR Product into Vector (Production of Recombinant Vector)

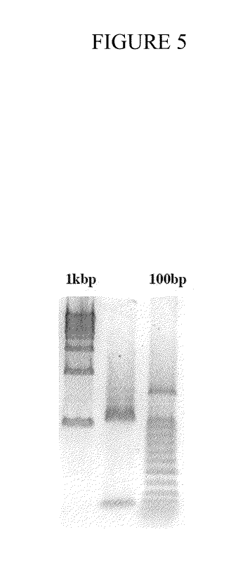

A vector (pUC18) was prepared by a known general method. In order to prevent the autocyclization of a plasmid DNA, dephosphorylation was carried out with phosphatase. When an insert was prepared, KOD-Plus with high precision was used as a polymerase for PCR. Looking at the product obtained after PCR shown in FIG. 5, a band was observed at 1 kbp, and thus it was found that PCR was carried out with no problems. Thereafter, the protecting group was dissociated by light irradiation, and phosphorylation was then carried out with a kinase. If deprotection was not carried out at this stage, there was the possibility that the kinase could not work well. In a case in which a primer whose 5′ end had previously been phosphorylated was used, the aforementioned operation could be omitted. The prepared vector was ligated to the insert, and was then transformed. As a result, colonies of transformant were observed. Thus, positive colonies were selected by colony PCR.

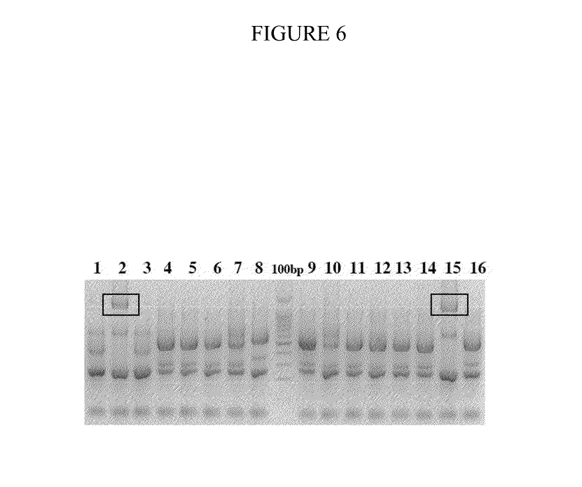

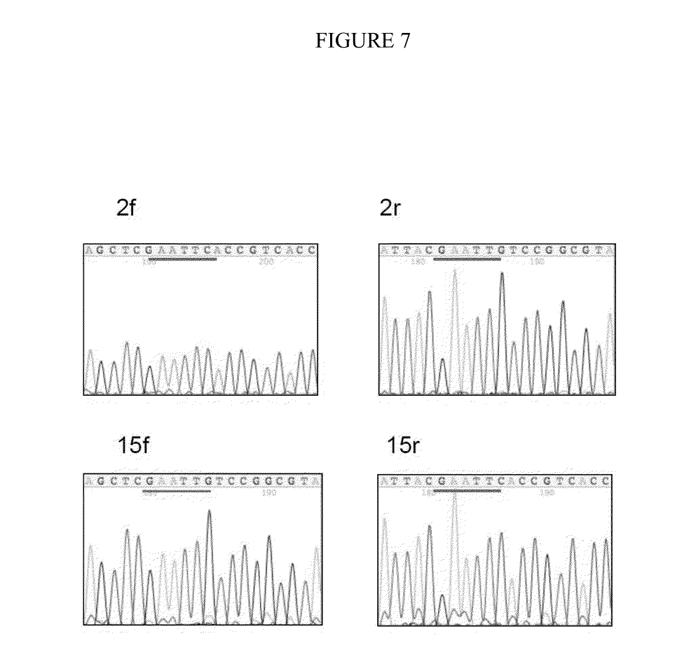

In FIG. 6, the plasmid DNAs of positive colonies in lane 2 and lane 15 were extracted, and the nucleotide sequences thereof were read. As a result, it could be confirmed that an insert was inserted therein in a form of interest (FIG. 7). When such insert was introduced into a single cleavage site, there were two ways of introducing the insert. That is, it was found that the insert was inserted in a desired direction in the case of lane 2, and that the insert was inserted in a reverse direction in the case of lane 15. In addition, it was also found that the sequence GAATTG was inserted on one side, and thus that one cleavage site recognized by EcoRI was broken. This method is characterized in that it is able to break a restriction site, and it is excellent in terms of usefulness and practical utility.

Thus, it was demonstrated that this method is able to precisely insert the insert prepared by LACE-PCR into a vector, so as to produce a recombinant vector.

### 3. Evaluation of LACE Primer by HPLC

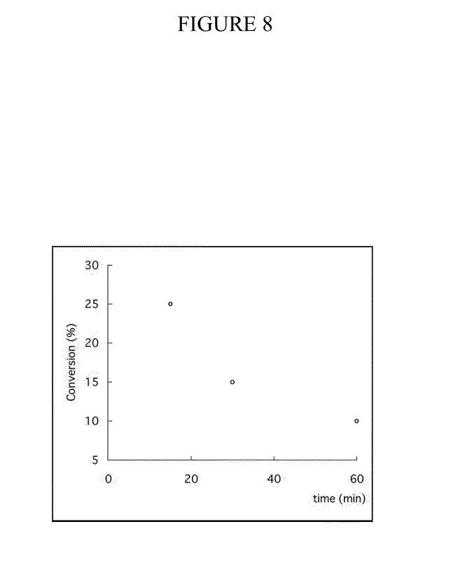

In order to evaluate the level of deprotection of a caged compound (a DNA having a base (T) protected by an NPP group) by UV irradiation, UV was applied to C3 and C4, and these primers were then analyzed by RP-HPLC. The results are shown in FIG. 8. In this analysis, since the detection wavelength was set at 260 nm. Thus, there was the possibility that the abundance ratio and the peak area ratio would be shifted because a compound with NPP as a protecting group and a compound without such NPP had different absorption coefficients. However, since the total peak area was not different so much before and after UV irradiation, the ratio was calculated, considering that such difference in absorption coefficients was negligible. From the graph shown in FIG. 8, it was found that 90% or more of NPP was deprotected by UV irradiation for 1 hour. This result shows that a sufficient amount of caged deprotection was carried out by UV irradiation for 1 hour. If it is assumed that 90% of two deprotections was completed, 80% thereof was a product of interest.

Since PCR conditions were relatively radical (heating to 90° C., etc.), there was possibility that NPP was dissociated by heat. Hence, C3 was incubated under the same conditions as PCR conditions, and it was then analyzed by HPLC. As a result, the percentage of compounds from which two NPPs acting as protecting groups were dissociated was 7.7%, the percentage of compounds from which one NPP was dissociated was 18.9%, and the percentage of compounds from which no NPP was dissociated was 73.4%. That is to say, the percentage of compounds, from which NPP on the 3′ side was not dissociated but remained, was considered to be approximately 85%. Since two primers were used in PCR, the percentage, in which a base at a position of interest was caged, was 70%.

Based on the aforementioned two factors, when NPP protection was used, the percentage of obtaining a PCR product having a 5′ protruding end of interest was considered to be approximately 50% to 60%.

4. Insertion of LACE-PCR Product into Two Cleavage Sites by EcoRI-HindIII

### 4-1. Designing and Synthesis of EcoRI-HindIII LACE Primer

As described above, genetic recombination operations were successfully carried out in the case of a system of one cleavage site. Next, the inventor made a second attempt to insert LACE-PCR products into a system of two cleavage sites. Basically, an experiment was carried out by the same method as that applied to the system of one cleavage site. Cl was used as a LACE primer, and it was designed to have a HindIII protruding end. In addition, in order to obtain an EcoRI protruding end, C3 was used. At this time, the LACE primer had only one caged protection. Thereby, whether or not a polymerase elongation reaction was terminated by one caged protection was simultaneously examined under PCR conditions. If the reaction is successfully carried out as intended, then the insert as shown in FIG. 9 is obtained.
4-2. Insertion of LACE-PCR Product into Vector (Production of Recombinant Vector)

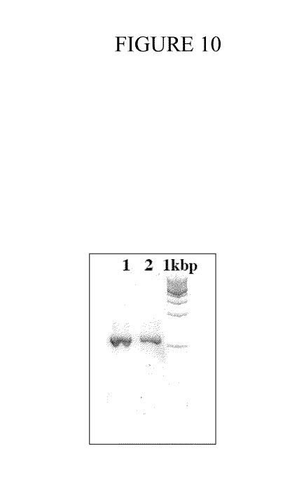

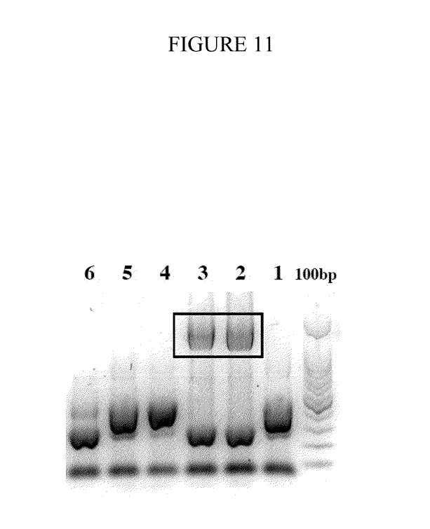

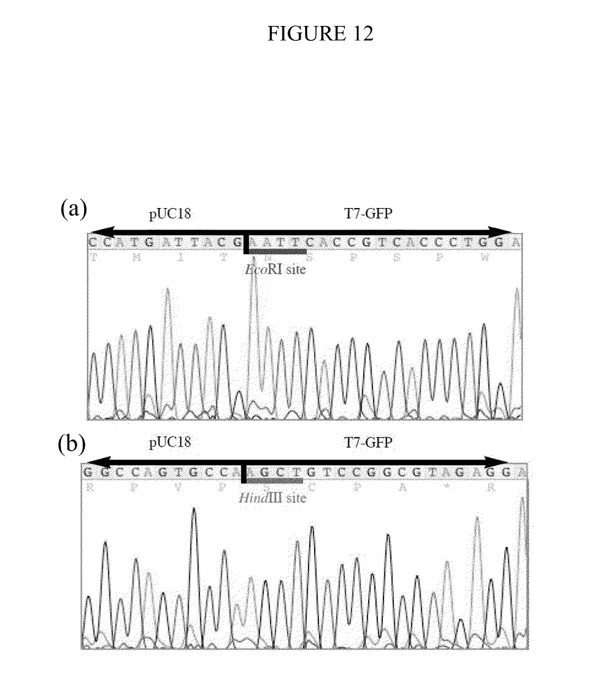

Basic procedures were the same as those described in Section 2 above. That is, pUC18 was reacted with HindIII at 37° C. for 4 hours, and it was further reacted with EcoRI at 37° C. for 16 hours, so as to prepare a vector having two cleavage sites. C1 and C3 were used as LACE-PCR primers, so as to prepare an insert (FIG. 10, lane 2). In addition, it could be confirmed from lane 1 that the prepared insert was not apparently damaged (for example, was not disconnected) by UV irradiation. The prepared insert was ligated to the vector, so that transformation was carried out. As a result, only 6 colonies of transformants were observed, and the two colonies thereof were found to be positive by colony PCR (lane 2 and lane 3 in FIG. 11). The plasmid DNAs of such positive transformants were extracted, and the nucleotide sequences thereof were read. As a result, it was found that the insert could be inserted in a form of interest (FIG. 12). Thereby, it could be confirmed that the prepared insert was sufficiently applicable to the system of two cleavage sites. Moreover, it was also demonstrated that only one caged protection is sufficient under these PCR conditions.

5. Insertion of LACE-PCR Product into One Cleavage Site by ARCUT

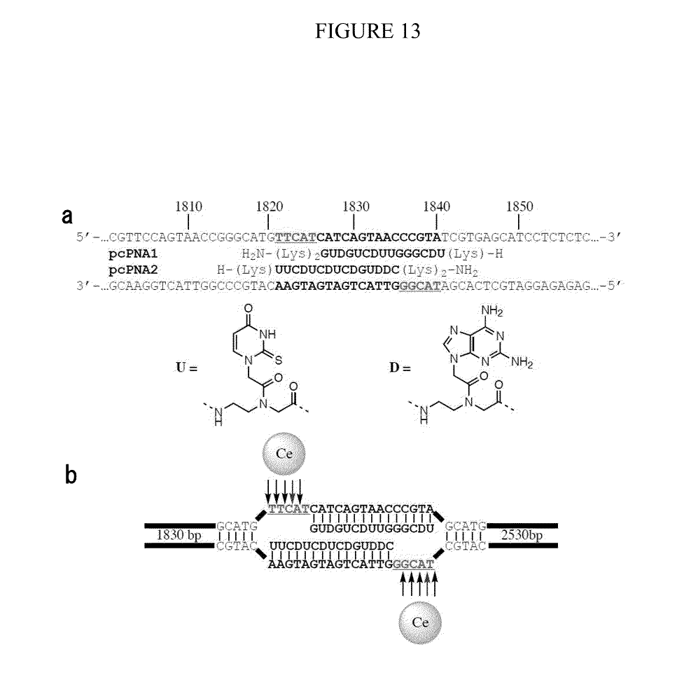

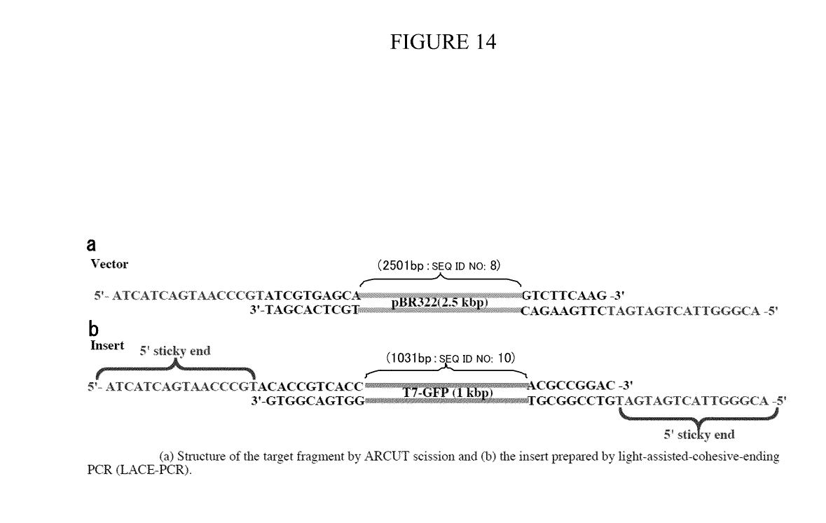

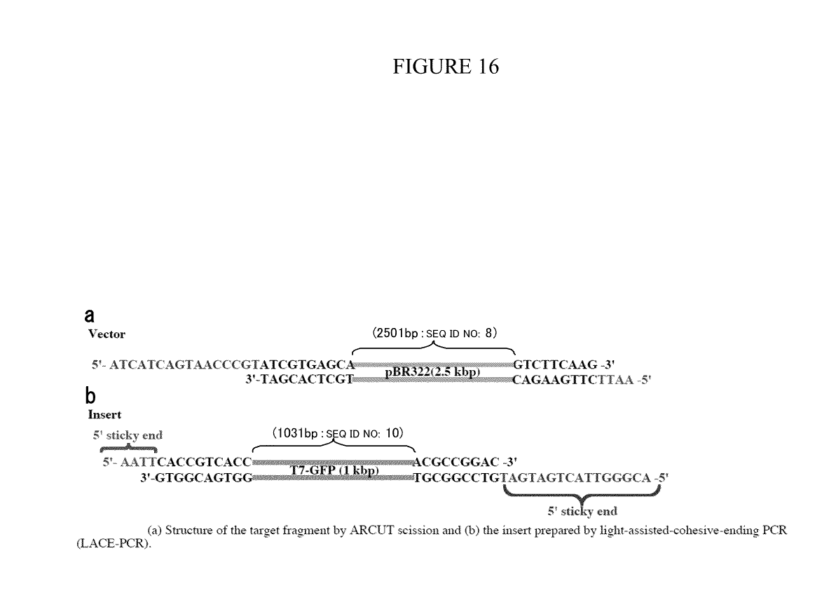

As described above, with regard to systems in which a vector was cleaved by a natural restriction enzyme, the insert prepared by LACE-PCR was successfully introduced into both the system of one cleavage site and the system of two cleavage sites. The greatest characteristic of this method is that a protruding end having any given sequence can be produced. Thus, the present inventor has thought of the possibility of directly ligating the insert to a protruding end generated after ARCUT cleavage, which does not exist in the nature. The inventor has attempted to directly insert the insert into a system in which pBR322 (Takara Bio Inc.; 4361 bp (SEQ ID NO: 7): GenBank Accession No. J01749) was cleaved by ARCUT at one cleavage site. As shown in FIG. 13a, the target cleavage site by ARCUT was determined around 1830 bp of the pBR322. The used pcPNA (pseudo-complementary peptide nucleic acid) is also shown in FIG. 13a. The cleavage of the vector by ARCUT occurs at any of the sites indicated with the arrows shown in FIG. 13b. This time, a vector fragment (FIG. 14a) generated as a result of the cleavage occurring at the site with the blue arrow (the second arrow from the right among the 5 arrows on both strands shown in FIG. 13b) was used as a target. With regard to the vector fragment shown in FIG. 14a, the nucleotide sequence of a portion indicated with pBR322 in the center (total 2501 bp: consisting of bases 1850-4350 in the nucleotide sequence shown in SEQ ID NO: 7) is shown in SEQ ID NO: 8 (the same applied to FIG. 16a). Thus, the C5 and C6 PCR primers in the above Scheme 1 were designed to produce an insert fragment having a protruding end shown in FIG. 14b. Using pQBI T7-GFP as a template, PCR and light irradiation were carried out by the same method as that in the Section 2-1 above, and as a result, a desired insert fragment (FIG. 14b) was synthesized and purified. An extremely important point for designing primers will be described below. Since the protruding end of ARCUT is extremely long, primers used in LACE-PCR also become long. The two primers comprise sequences complementary to each other, and thus they easily form a primer dimer. Hence, as with C5 and C6, T^{NPP} has been introduced into such a portion complementary to each other to inhibit the interaction between the primers, and thus, the primers were designed so as not to form a primer dimer.

5-2. Single-Site Cleavage of pBR322 bp ARCUT and Purification of cleavage Fragment

A fragment obtained by cleaving supercoiled pBR322DNA by ARCUT was purified using gel, so as to obtain a linearized fragment. Thereafter, an excessive amount of oligo DNA complementary to PNA was added thereto to remove PNA, and a phosphatase treatment was then performed for dephosphorylation.

5-3. Insertion of LACE-PCR Product into Vector (Production of Recombinant Vector)

It was confirmed that the insert was amplified by LACE-PCR as designed. Thus, after completion of the caged deprotection, the resultant was treated with kinase, and insertion of the product into the vector linearized by ARCUT was then attempted. However, in conclusion, this system was not successful at all. The main reason is considered that, since the protruding end is long, autocyclization occurs more easily than a system cleaved only at a single site by restriction enzyme. The amount of the insert was increased from 50 equivalents to 200 equivalents with respect to the amount of the vector, and ligation was carried out. However, no positive colonies could be obtained by colony PCR in all the cases. Moreover, even in a case in which Escherichia coli was transformed without ligation reactions, colonies were increased. As a result, it was considered that the insert could be sufficiently inserted into this system even without ligation reactions.

6. Insertion of LACE-PCR Product into Two Cleavage Sites by EcoRI-ARCUT

### 6-1. Designing of EcoRI-ARCUT LACE Primer

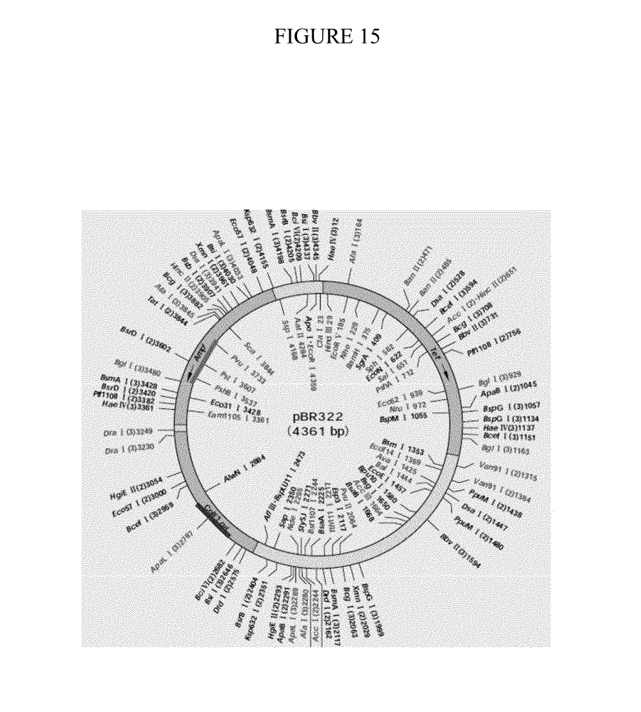

Two fragments, 1830 bp and 2530 bp, can be obtained by cleaving the pBR322 linearized by EcoRI at a site around 1830 bp. Of the two fragments, the 2530 bp fragment has a drug resistance gene (Amp) and a replication origin (ori), and thus it acts as a vector (FIG. 15). Hence, using the previously synthesized C3 and C6 as LACE primers, a fragment having a protruding end as shown in FIG. 16b was produced, and the insert was then inserted into an ARCUT cleavage fragment as shown in FIG. 16a.

6-2. Two-Site Cleavage of pBR322 by EcoRI and ARCUT, and Purification of Cleavage Fragment

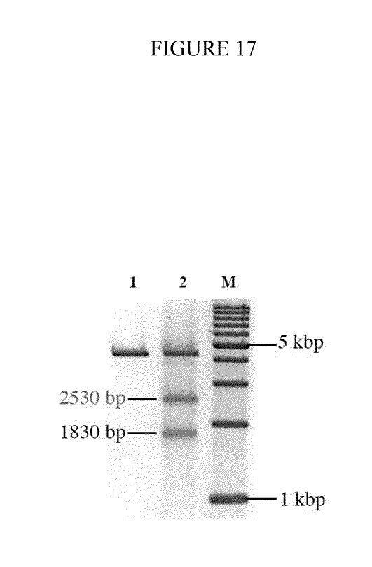

The pBR322 linearized by EcoRI was further cleaved by ARCUT, and the resultant was then purified in the same manner as in the Section 4-2 above. FIG. 17 shows the results of electrophoresis performed after the cleavage of pBR322 (lane 1: cleaved only by EcoRI; lane 2: cleaved by both EcoRI and ARCUT).

6-3. Insertion of LACE-PCR Product into Vector (Production of Recombinant Vector)

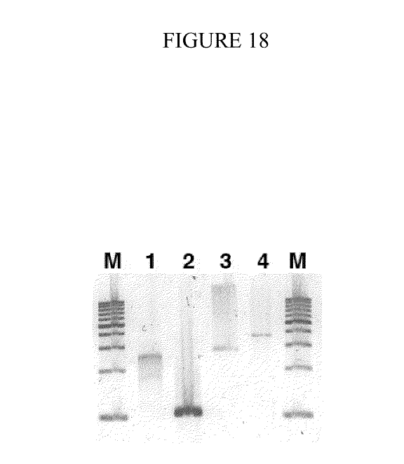

It was confirmed that the insert was amplified by LACE-PCR as designed. Thus, after completion of the caged deprotection, the resultant was treated with kinase. Thereafter, the vector prepared in the Section 5-2 above was mixed with the insert at a mixing ratio of 1:40 to ligate them, and the obtained ligate was then introduced into DH5α so as to transform it. Since generation of colonies was confirmed, colony PCR was carried out in the same manner as that described above, and the plasmid DNA of positive colonies was extracted. The plasmid DNA was cleaved at a single site by EcoRI, so that the size of the extracted plasmid DNA was confirmed (FIG. 18, lane 4).

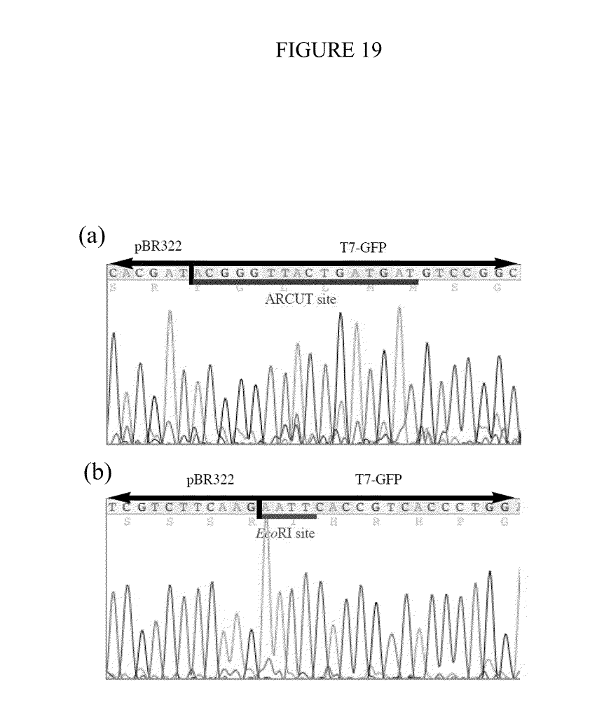

The nucleotide sequence of the plasmid DNA was determined by the same method as that described above. As a result, as shown in FIG. 19, it was found that a portion cleaved by EcoRI and a portion cleaved by ARCUT could be ligated at positions of interest.

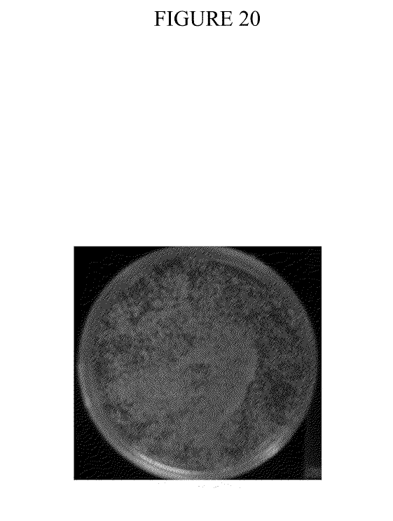

Furthermore, the present plasmid was introduced into BL21-Gold (DE3) having a T7 RNA polymerase so as to transform it. As a result, it could be confirmed that GFP was expressed (FIG. 20).

### 7. Conclusion and Consideration

To sum up the present examples, when a caged DNA was used as a template, polymerase was not able to promote an elongation reaction (a complementary strand synthetic reaction) after a base protected by a protecting group, and any given 5′ end protruding structure was formed. Since such caged protection could be easily deprotected by UV irradiation, this technique was applied to genetic recombination technology. That is, PCR was carried out using a caged DNA as a primer, so that a 5′ protruding end could be successfully prepared (LACE-PCR). The insert prepared by LACE-PCR was successfully inserted into a vector subjected to a restriction enzyme digestion and a vector digested by ARCUT. Thereby, the inventor succeeded in performing ligation on an ARCUT inserted fragment at a position of interest without using Oligojoint. As a result of the establishment of this technique, this technique was considered to be applied, for example, to genetic engineering using ARCUT. In recent years, the chemical synthesis of bacterial genome has been succeeded. In this chemical synthesis as well, one key point was production of a specific protruding end. It was considered that gene synthesis, DNA-overhang cloning (DOC) and the like could be accomplished by applying the method of the present invention.

### Example 2

### Genetic Recombination that Utilizes LACE-PCR Method without Using Ligase (Ligation-Independent Cloning)

1. Production of GFP-BFP Gene that Involves Insertion into Plasmid Comprising GFP Gene

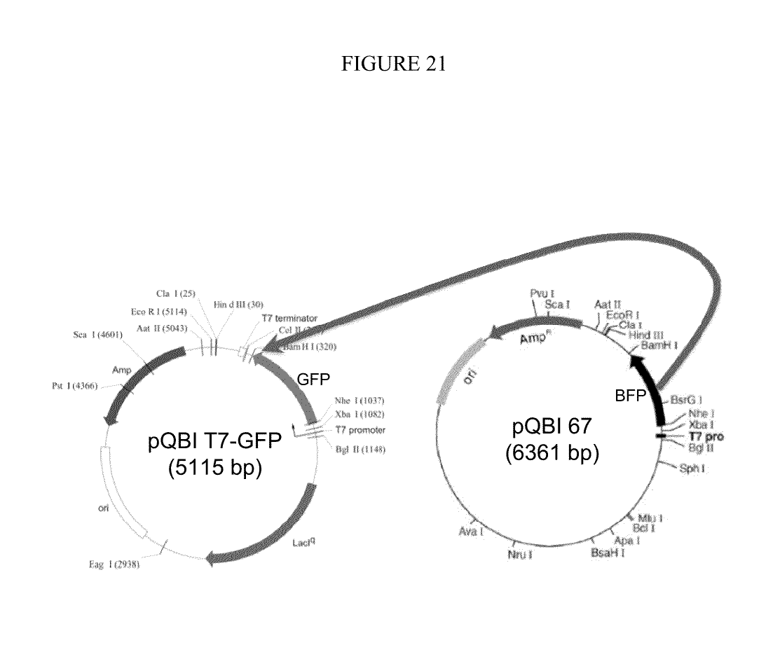

It has been known that, when two fragments having relatively long sticky ends are used, transformation can be carried out by utilizing the repair mechanism of Escherichia coli without ligation. An entire-length of a pQBI T7-GFP vector (Qbiogene; 5115 bp (SEQ ID NO: 9); FIG. 21, left) having a long sticky end consisting of approximately 10 bases, which had been amplified by LACE-PCR, was allowed to bind to a BFP fragment in a pQBI 67 vector (Qbiogene; 6361 bp; FIG. 21, right), which had also been amplified by LACE-PCR, thereby constructing a novel vector encoding a GFP-BFP fusion protein (see FIG. 21).

### 2. Designing and Synthesis of LACE Primers

2-1. Primers Used in Amplification of pQBI-T7 GFP Vector

In order to amplify an entire-length of a pQBI-T7 GFP vector, the following PCR primers, C7 and C8, were synthesized.

(SEQ ID NO: 11)

| | |
|---|---|
| C7 | 5′-pACCGCCACCT^{NPP}CCGTTGTACAGTTCATCCATGCC-3′ |

(SEQ ID NO: 12)

| | |
|---|---|
| C8 | 5′-pAGGATCCGGCT^{NPP}GCTAACAAAGCCCGAAAGGAAGC-3′ |

C7 and C8 are primers for amplifying the entire nucl [symbol] obtained by removing bases 314 to 328 from the pQBI T7-GFP vector. Since the thus removed region comprises the step codon of the GFP gene, if a DNA fragment comprising a foreign protein gene is inserted, a fusion protein with GFP is translated (generated). To the 5′ end side from the T^{NPP} of the primer that would become a sticky end as a result of LACE-PCR, 9 bases encoding a glycine linker (C7) or 10 bases identical to bases 315 to 324 of the pQBI T7-GFP vector (C8) was added.

### 2-2. Primers Used in Amplification of BFP Gene

In order to amplify a BFP gene encoded by a pQBI 67 vector by LACE-PCR, the following PCR primers, C9 and C10, were synthesized.

| | | |
|---|---|---|
| C9 | 5′-pAGCCGGATCCT^{NPP}CAGTTGTACAGTTCATCCATGCC-3′ | [symbol] |
| C10 | 5′-pAGGTGGCGGT^{NPP}GGCAAAGGAGAAGAACTCTTCACTGG-3′ | [symbol] |

C9 and C10 are primers for amplifying a nucleotide sequence region (701 bp; SEQ ID NO: 15) encoding a main body of BFP consisting of bases 335 to 1035 of the pQBI 67 vector. To the 5′ end side from the T^{NPP} of the primer that would become a sticky end as a result of LACE-PCR, 9 bases encoding a glycine linker (C10) or 10 bases identical to bases 315 to 324 of the pQBI T7-GFP vector (C9) was added.

### 2-3. LACE-PCR

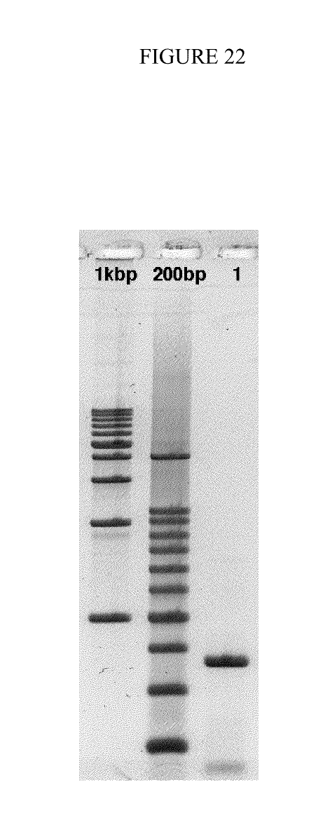

LACE-PCR was carried out using the above-described primers. For amplification of the vector fragments C7 and C8, commercially available Pfu ultra was used. For amplification of the BFP inserts C9 and C10, Pfu turbo was used as polymerase in PCR. After completion of the PCR, the PCR product was digested with Dpn I for digesting a methylated DNA at 37° C. overnight, and the amplified fragment was simply purified using a commercially available kit, so as to obtain an aqueous solution (TE buffer) of each fragment. Subsequently, the NPP protecting group was deprotected by UV irradiation. The results obtained by subjecting the amplified fragment to agarose electrophoresis are shown in FIG. 22. In the case of an amplified fragment supplied to lane 1, a band was observed between a 600-bp fragment and a 800-bp fragment on a ladder of 200-bp units, and thus it was confirmed that a 710-bp fragment was amplified as designed.

2-4. Transformation of Escherichia coli with LACE-PCR Product

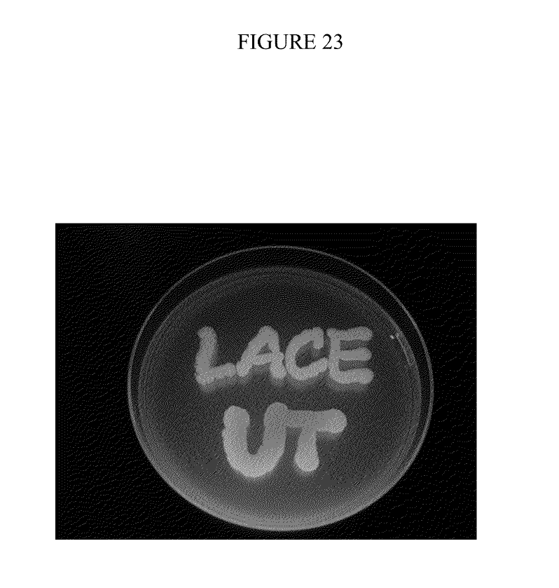

A solution corresponding to 50 ng of a vector fragment and a solution corresponding to 24 ng of a BFP insert (approximately 3 equivalents relative to mole; total amount: 2 μl) were mixed to each other, and the mixed solution was then incubated at 37° C. for 1 hour. Thereafter, the reaction solution was added to a solution of JM109 Escherichia coli strain without a ligation reaction, so that the Escherichia coli was transformed. This Escherichia coli solution was cultured on a plate containing 50 μg of carbenicillin overnight. As a result, 6 colonies were obtained. Colony PCR was carried out, and as a result, it was confirmed that 5 colonies were recombinant bodies of interest. Each colony was subjected to liquid culture, and its plasmid was extracted. Thereafter, a BL21(DE3) strain was transformed with each plasmid. As a result of culture on a plate containing 50 μg of carbenicillin, the generated colony emitted dark green fluorescence. FIG. 23 shows the color of the fluorescence of this Escherichia coli strain. The two letters “UT” indicate the fluorescence of the recombinant body produced in the present example. The color of the fluorescence was different from either the letter “L” having only the GFP gene or the letters “ACE” having only the BFP gene.

### 2-5. Confirmation of Construction of Recombinant Vector as Designed

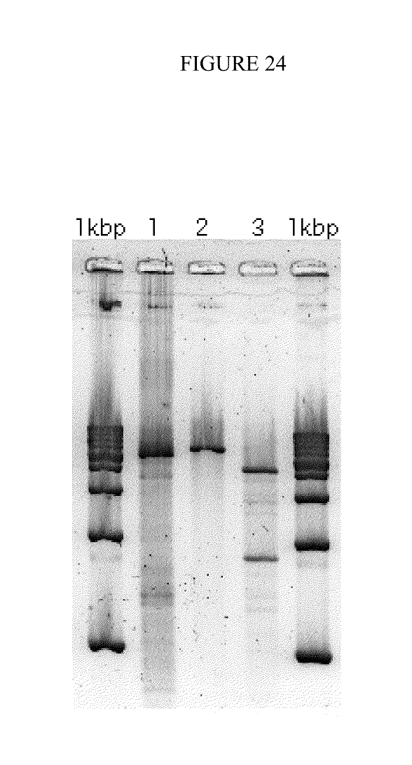

The extracted plasmid was cleaved by restriction enzymes, and whether it had a correct length was then confirmed. The results of agarose gel electrophoresis are shown in FIG. 24. The extracted plasmid (lane 2), which had been converted to a straight chain by a digestion with Eco RI, clearly had a mobility on electrophoresis that was lower than that of a vector fragment amplified by LACE-PCR (lane 1; 5125 bp). Thus, it was found that the BFP fragment was correctly inserted. This result could also be confirmed by the fact that the predicted 1862-bp and 3975-bp bands were clearly observed also in lane 3 involving two-site cleavage by Eco RI and Nhe I.

### 2-6. Confirmation of Protein Expressed by Recombinant Vector

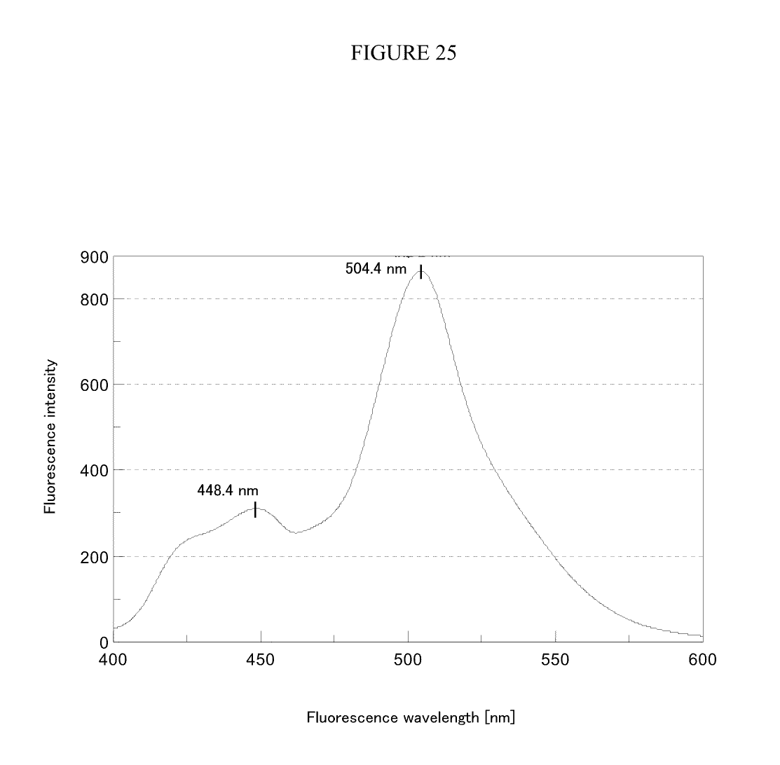

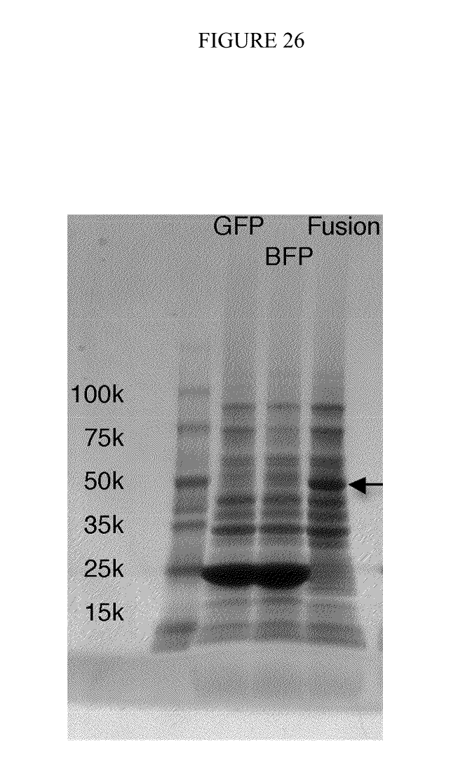

In order to confirm that a protein expressed by the recombinant vector is correctly a GFP-BFP fusion protein, the transformed Escherichia coli was subjected to liquid culture, and was then subjected to a bacteriolytic treatment. The fluorescence of the bacteriolytic solution was measured. As a result, two fluorescence peaks, 448 nm derived from BFP and 504 nm derived from GFP, were confirmed (FIG. 25). In addition, the bacteriolytic solution was analyzed by SDS-PAGE. As a result, it was confirmed that a novel approximately 50 kDa protein (a GFP-BFP fusion protein; Fusion), which differed from either BFP (26.9 kDa) or GFP (26.9 kDa), was strongly expressed (FIG. 26).

### Example 3

### Synthesis of Thymine Protected by 2-(2-nitrophenyl)propyloxymethyl Group and Amidite Monomer

An amidite monomer to be introduced into thymine protected by a 2-(2-nitrophenyl)propyloxymethyl group was synthesized according to the following Scheme 2.

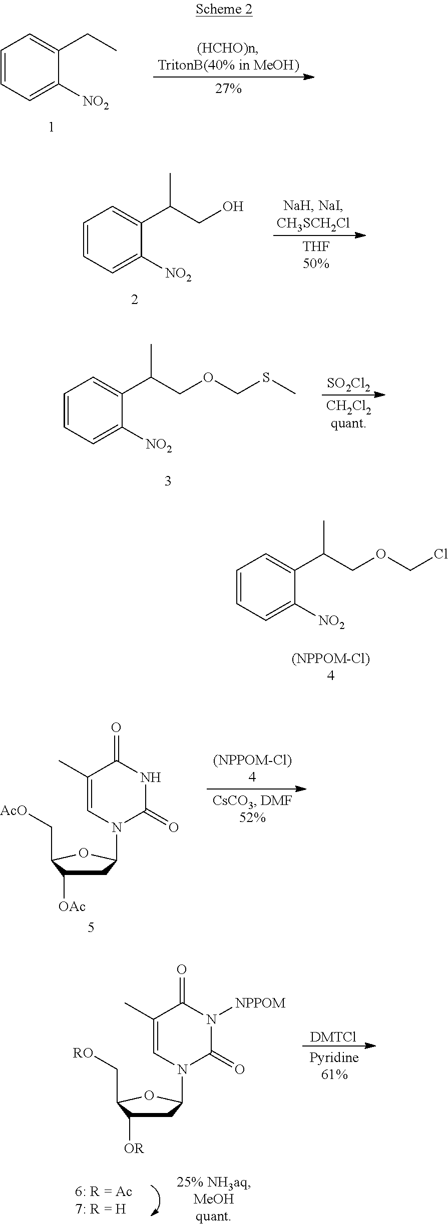

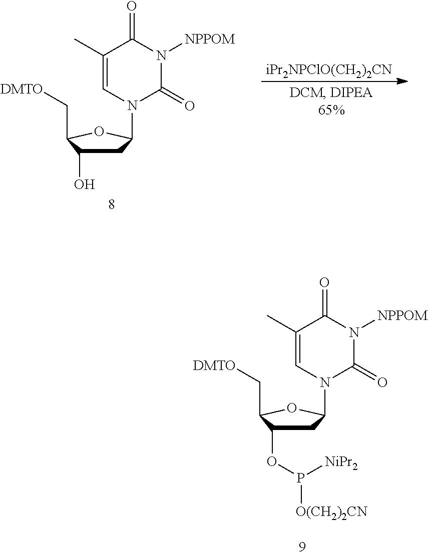

Compounds (Compounds 2-9) shown in the Scheme 2 were specifically synthesized as follows.

### Synthesis of 2-(2-nitrophenyl)propanol (Compound 2)

Compound 1 (2-ethylnitrobenzene) (6.0 g, 40 mmol) and (HCHO)ₙ (1.5 g, 40 mmol) were added into TritonB (40% MeOH solution) (17 g), and the obtained mixture was then heated under reflux for 7 hours. Thereafter, the solvent was distilled away, and the resultant was then re-dissolved in ethyl acetate. The organic layer was washed with 5% hydrochloric acid. The organic layer was dried over sodium sulfate, and was then purified by silica gel chromatography using hexane:ethyl acetate (3:1) as a developing solvent, so as to obtain 1.95 g of a reddish violet oily product of interest (Compound 2) (10.7 mmol, yield: 27%).

¹H NMR (CDCl₃) δ=1.34 (3H, d, CH₃), 3.50-3.58 (1H, m, CH), 3.77-3.86 (2H, m, CH₂), 7.36 (1H, t, Ar—H), 7.50 (1H, d, Ar—H), 7.58 (1H, t, Ar—H), 7.76 (1H, d, Ar—H)

### Synthesis of 2-[(2-nitrophenyl)-(methylthio)methoxy]-propane (Compound 3)

Compound 2 (1.95 g, 10.7 mmol) was dissolved in 20 mL of THF, and the obtained solution was then cooled at 0° C. in a nitrogen atmosphere. While stirring, sodium hydride (60%) (0.6 g) was added to the reaction solution. The obtained mixture was stirred for 45 minutes, and sodium iodide (1.62 g, 10.7 mmol) was then added to the reaction solution. Subsequently, chloromethyl methyl sulfide (ClCH₂SCH₃; 1.29 g, 13.35 mmol) was added thereto. The obtained mixture was stirred under cooling on ice for 4 hours, and the temperature of the reaction solution was then returned to room temperature. Thereafter, the solvent was distilled away. 30 mL of water was added to the residue, and was then extracted with ether. The organic layer was dried over sodium sulfate, and was then purified by silica gel chromatography using dichloromethane as a developing solvent, so as to obtain 1.32 g of a brown oily product of interest (Compound 3) (5.43 mmol, yield: 50%).

¹H NMR (CDCl₃) δ=1.36 (3H, d, CH₃), 2.00 (3H, s, CH₃), 3.63-3.76 (3H, m, CH, CH₂), 4.58 (2H, d, CH₂), 7.34 (1H, t, Ar—H), 7.52 (2H, m, Ar—H), 7.75 (1H, d, Ar—H)

### Synthesis of 2-[(2-nitrophenyl)-chloromethoxy]-propane (Compound 4)

Compound 3 (1.32 g, 5.43 mmol) was dissolved in 20 mL of dichloromethane. While the obtained solution was stirred at 0° C. in a nitrogen atmosphere, SO₂Cl₂ (0.94 g, 6.96 mmol) was added to the solution. The obtained mixture was stirred under cooling on ice for 4 hours, and the temperature of the reaction solution was then returned to room temperature. Thereafter, an excessive amount of SO₂Cl₂ was distilled away under reduced pressure. The reaction was quantitative. The obtained compound (Compound 4) was directly used in the subsequent reaction without purification.

¹H NMR (CDCl₃) δ=1.37 (3H, d, CH₃), 3.66 (1H, m, CH), 3.81 (2H, m, CH₂), 5.43 (2H, q, CH₂), 7.36 (1H, t, Ar—H), 7.48 (1H, d, Ar—H), 7.57 (1H, t, Ar—H), 7.76 (1H, d, Ar—H)

### Synthesis of Thymidine Acetate (Compound 5)

Thymidine (2.93 g, 12.1 mmol) and DMAP (40 mg, 0.327 mmol) were dissolved in 60 mL of acetic anhydride in a nitrogen atmosphere, and 30 mL of dry pyridine was then added to the solution, followed by stirring at room temperature overnight. Thereafter, pyridine and acetic anhydride were distilled away under reduced pressure, and 100 mL of chloroform was then added to the residue. The organic layer was washed with 100 mL of water. The organic layer was dried over anhydrous sodium sulfate, and was then purified by silica gel chromatography using hexane:ethyl acetate (1:3) as a developing solvent, so as to obtain 2.82 g of a product of interest (Compound 5) (8.64 mmol, yield: 71%).

¹H NMR (CDCl₃) δ=1.94 (3H, s, CH₃), 2.10-2.22 (8H, m, 2×CH₃, CH₂), 2.46 (1H, m, CH), 4.36 (2H, dd, CH₂), 5.22 (1H, d, CH), 6.32 (1H, q, CH), 7.29 (1H, s, CH), 8.65 (1H, s, NH)

### Synthesis of 3-N-[2-(2-nitrophenyl)-propyloxymethyl]thymidine acetate (Compound 6)

Compound 5 was dissolved in 20 mL of DMF, and cesium carbonate was then added to the solution, followed by stirring at room temperature for 1 hour. Subsequently, the reaction solution was cooled on ice, and Compound 4 (1.26 g, 5.47 mmol) dissolved in DMF was then added dropwise thereto. The solvent was distilled away under reduced pressure, and ethyl acetate was then added to the residue. The organic layer was washed with 30 mL of water twice, and then with a saturated sodium bicarbonate solution once. It was then dried over sodium sulfate. The solvent was distilled away under reduced pressure, and the residue was then purified by silica gel chromatography using hexane:ethyl acetate (1:2) as a developing solvent, so as to obtain 1.50 g of a product of interest (Compound 6) (2.89 mmol, yield: 52%).

¹H NMR (CDCl₃) δ=1.31 (3H, dd, CH₃), 1.93 (3H, d, CH₃), 2.11 (8H, m, 2×CH₃, CH₂), 3.57 (1H, m, CH), 3.74-3.88 (2H, m, CH₂), 4.25 (1H, s, CH), 4.34 (2H, m, CH₂), 5.21 (1H, d, CH), 5.36 (2H, m, CH₂), 7.28 (1H, s, CH), 7.31 (1H, t, Ar—H), 7.46 (1H, d, Ar—H), 7.51 (1H, t, Ar—H), 7.71 (1H, t, Ar—H)

### Synthesis of 3-N-[2-(2-nitrophenyl)-propyloxymethyl]thymidine (Compound 7)

Compound 6 (1.50 g, 2.89 mmol) was dissolved in 20 mL of methanol, and 8 mL of concentrated ammonia water was then added to the solution, followed by stirring at room temperature for 1 hour. Thereafter, the solvent was distilled away, so as to quantitatively obtain a product of interest (Compound 7)

¹H NMR (CDCl₃) δ=1.31 (3H, d, CH₃), 1.90 (3H, d, CH₃), 2.31-2.42 (2H, m, CH₂), 3.55-3.65 (1H, m, CH), 3.75-3.95 (4H, m, 2×CH₂), 3.97 (1H, q, CH), 4.55 (1H, m, CH), 5.34 (2H, m, CH₂), 7.31 (1H, t, CH), 7.43 (2H, t, 2×Ar—H), 7.52 (1H, t, Ar—H), 7.70 (1H, dd, Ar—H)

### Synthesis of 3-N-[2-(2-nitrophenyl)-propyloxymethyl]thymidine dimethoxytrityl ether (Compound 8)

Compound 7 (1.26 g, 2.89 mmol) and dimethoxytrityl chloride (1.19 g, 3.51 mmol) were dissolved in 20 mL of pyridine, and the obtained mixture was then stirred in a nitrogen atmosphere at room temperature overnight. Thereafter, the solvent was distilled away under reduced pressure, and the residue was then dissolved in 30 mL of ethyl acetate. The organic layer was washed with a saturated saline, and was then dried over sodium sulfate. Thereafter, the solvent was distilled away under reduced pressure. The residue was purified by silica gel chromatography using 3% triethylamine-containing hexane:ethyl acetate (1:1) as a developing solvent, so as to obtain 1.31 g of a product of interest (Compound 8) (1.77 mmol, yield: 61%)

¹H NMR (CDCl₃) δ=1.31 (3H, d, CH₃), 1.92 (3H, d, CH₃), 2.28-2.50 (2H, m, CH₂), 3.56 (1H, m, CH), 3.76-3.90 (10H, m, 2×CH₂, 2×CH₃), 3.92 (1H, m, CH), 4.56 (1H, m, CH), 5.37 (2H, m, CH₂), 6.16 (1H, d, CH), 6.84 (4H, d, 4×Ar—H), 7.17 (3H, d, 3×Ar—H), 7.27-7.36 (7H, m, 6×Ar—H, CH), 7.36 (1H, d, Ar—H), 7.47 (1H, d, Ar—H), 7.51 (1H, q, Ar—H), 7.71 (1H, d, Ar—H)

ESI-MS [M+Na]⁺ m/z=760.8

### Synthesis of 3-N-[2-(2-nitrophenyl)-propyloxymethyl]thymidine phosphoramidite (Compound 9)

Compound 8 (0.30 g, 0.406 mmol) was dissolved in 2 mL of dry acetonitrile in a nitrogen atmosphere. Diisopropylethylamine and 2-cyanoethyl-diisopropyl-chlorophosphoramide (200 μL, 0.912 mmol) were added to the solution, and the obtained mixture was then stirred at room temperature for 1.5 hours. Thereafter, the solvent was distilled away under reduced pressure, and a saturated sodium bicarbonate solution and ethyl acetate were then added to the residue to extract the organic layer. The organic layer was washed with a saturated saline, and was then dried over sodium sulfate. Thereafter, the solvent was distilled away under reduced pressure. The residue was purified by silica gel chromatography using 3% triethylamine-containing chloroform:methanol (20:1) as a developing solvent, so as to obtain 0.25 g of a product of interest (Compound 9) (0.26 mmol, yield: 65%).

ESI-MS [M+Na]⁺ m/z=961.7

### INDUSTRIAL APPLICABILITY

According to the present invention, there can be provided a method for preparing a DNA fragment, in which a desired double-stranded DNA fragment having a sticky end is directly and easily obtained from an amplification product (an amplified fragment) after PCR without a restriction enzyme digestion. In addition, according to the present invention, there can be provided a novel PCR primer that can be used in the above-described preparation method. Moreover, according to the present invention, there can be provided a genetic recombination method using the DNA fragment obtained by the above-described preparation method.

According to the preparation method of the present invention, either a DNA fragment having a sticky end consisting of a nucleotide sequence identical to that of a sticky end obtained by a restriction enzyme digestion, or a DNA fragment having a sticky end consisting of a nucleotide sequence different from that of a sticky end obtained by a restriction enzyme digestion, can be easily obtained as a double-stranded DNA fragment having a sticky end. Herein, with regard to the latter DNA fragment, the base length of a sticky end portion thereof (a single-stranded DNA portion) can be set at a desired length by arbitrarily designing the base length of a portion that does not bind to a template DNA in a PCR primer. Thus, such base length is not particularly limited. Accordingly, it is easily possible to prepare a DNA fragment whose specificity and binding efficiency have been improved to the desired levels, in terms of the binding of the DNA fragment to a host DNA. Furthermore, when a DNA fragment having a sticky end portion with a long base length is used in genetic recombination, for example, the DNA fragment is able to bind to a host DNA only by mixing them, without using a conventional DNA ligase such as a ligase. Therefore, the present invention can also provide a genetic recombination method that is excellent in terms of simplicity, economic efficiency and practical utility.

### SEQUENCE LISTING FREE TEXT

SEQ ID NO: 1 synthetic DNA
SEQ ID NO: 1 t is modified with a 2-(2-nitrophenyl)propyl group (NPP group) (position 4).
SEQ ID NO: 2 synthetic DNA
SEQ ID NO: 3 synthetic DNA
SEQ ID NO: 3 t is modified with a 2-(2-nitrophenyl)propyl group (NPP group) (position 3).
SEQ ID NO: 3 t is modified with a 2-(2-nitrophenyl)propyl group (NPP group) (position 4).
SEQ ID NO: 4 synthetic DNA
SEQ ID NO: 4 t is modified with a 2-(2-nitrophenyl)propyl group (NPP group) (position 3).
SEQ ID NO: 4 t is modified with a 2-(2-nitrophenyl)propyl group (NPP group) (position 4).
SEQ ID NO: 5 synthetic DNA
SEQ ID NO: 5 t is modified with a 2-(2-nitrophenyl)propyl group (NPP group) (position 9).
SEQ ID NO: 5 t is modified with a 2-(2-nitrophenyl)propyl group (NPP group) (position 16).
SEQ ID NO: 6 synthetic DNA
SEQ ID NO: 6 t is modified with a 2-(2-nitrophenyl)propyl group (NPP group) (position 8).
SEQ ID NO: 6 t is modified with a 2-(2-nitrophenyl)propyl group (NPP group) (position 16).
SEQ ID NO: 7 cloning vector pBR322
SEQ ID NO: 8 cloning vector pBR322
SEQ ID NO: 9 cloning vector pQBI T7-GFP
SEQ ID NO: 10 cloning vector pQBI T7-GFP
SEQ ID NO: 11 t is modified with a 2-(2-nitrophenyl)propyl group (NPP group) (position 10).
SEQ ID NO: 11 t is modified with a 2-(2-nitrophenyl)propyl group (NPP group) (position 10).
SEQ ID NO: 12 t is modified with a 2-(2-nitrophenyl)propyl group (NPP group) (position 11).
SEQ ID NO: 12 t is modified with a 2-(2-nitrophenyl)propyl group (NPP group) (position 11).
SEQ ID NO: 13 t is modified with a 2-(2-nitrophenyl)propyl group (NPP group) (position 11).
SEQ ID NO: 13 t is modified with a 2-(2-nitrophenyl)propyl group (NPP group) (position 11).
SEQ ID NO: 14 t is modified with a 2-(2-nitrophenyl)propyl group (NPP group) (position 10).
SEQ ID NO: 14 t is modified with a 2-(2-nitrophenyl)propyl group (NPP group) (position 10).
SEQ ID NO: 15 cloning vector pQBI67 (BFP gene)

1. A primer used for PCR, which is composed of a complementary DNA portion consisting of a nucleotide sequence complementarily binding to an amplification target region in a template DNA and a non-complementary DNA portion consisting of a nucleotide sequence that links to the 5′ end of the complementary DNA portion but does not complementarily bind to the amplification target sequence,
wherein at least a base corresponding to the 3′ end in the nucleotide sequence of the non-complementary DNA portion is modified with a protecting group capable of terminating the progression of DNA replication catalyzed by a DNA polymerase.

2. The primer according to claim 1, wherein the protecting group can be dissociated from the modified base by a light irradiation treatment, an alkali treatment, an acid treatment, an oxidation treatment, a reduction treatment, a desilylation treatment, a heat treatment, an esterase treatment or a phosphatase treatment.

3. The primer according to claim 2, wherein the protecting group that can be dissociated from the modified base by the light irradiation treatment is a 2-(2-nitrophenyl)propyl group, a 2-(2-nitrophenyl)propyloxymethyl group, a 1-(2-nitrophenyl)ethyl group or a 6-nitropiperonyloxymethyl group.

4. The primer according to claim 2, wherein the protecting group that can be dissociated from the modified base by the alkali treatment is an isobutyryl group, a benzoyl group or an acetoxymethyl group.

5. The primer according to claim 2, wherein the protecting group that can be dissociated from the modified base by the acid treatment is a trityl group or a methoxy derivative thereof.

6. The primer according to claim 2, wherein the protecting group that can be dissociated from the modified base by the oxidation treatment is an allyloxymethyl group, a dimethoxybenzyloxymethyl group or a trimethoxybenzyloxymethyl group.

7. The primer according to claim 2, wherein the protecting group that can be dissociated from the modified base by the reduction treatment is a benzyloxymethyl group, or a benzyloxymethyl group substituted with any given substituent.

8. The primer according to claim 2, wherein the protecting group that can be dissociated from the modified base by the desilylation treatment is a t-butyldimethoxysilyloxymethyl group or a t-butyldiphenylsilyloxymethyl group.

9. The primer according to claim 2, wherein the protecting group that can be dissociated from the modified base by the heat treatment is an isocyanate group.

10. The primer according to claim 2, wherein the protecting group that can be dissociated from the modified base by the esterase treatment is an acetoxymethyl group.

11. The primer according to claim 2, wherein the protecting group that can be dissociated from the modified base by the phosphatase treatment is a methyl phosphate group.

12. The primer according to any one of claims 3 to 11, wherein at least a base corresponding to the 3′ end in the nucleotide sequence of the non-complementary DNA portion is thymine or guanine.

13. The primer according to claim 1, wherein the non-complementary DNA portion is 1 to 100 bases in length.

14. A method for preparing a DNA fragment having a sticky end, which comprises:
(i) a step of performing a PCR reaction using a template DNA and the primer according to claim 1 to obtain an amplified DNA fragment; and
(ii) a step of performing a prescribed treatment on the amplified DNA fragment to dissociate a protecting group from the fragment.

15. The method according to claim 14, wherein the prescribed treatment for dissociating the protecting group from the modified base is a light irradiation treatment, an alkali treatment, an acid treatment, an oxidation treatment, a reduction treatment, a desilylation treatment, a heat treatment, an esterase treatment or a phosphatase treatment.

16. The method according to claim 14, wherein the sticky end has the same nucleotide sequence as that of a sticky end obtained by a restriction enzyme digestion.

17. The method according to claim 14, wherein the sticky end has a nucleotide sequence different from that of a sticky end obtained by a restriction enzyme digestion.

18. A genetic recombination method, which comprises a step of binding the DNA fragment obtained by the method according to any one of claims 14 to 17 to a host DNA.

19. The method according to claim 18, wherein the binding operation is carried out without using a DNA ligase.

20. A substituent-introducing agent comprising a compound represented by the following formula (I′): 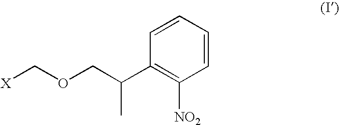
[wherein, in the formula (I′), X represents Cl, I, Br, p-toluenesulfonic acid ester or sulfuric acid ester].

21. A biomolecule having a 2-(2-nitrophenyl)propyloxymethyl group.

22. The biomolecule according to claim 21, which is represented by the following formula (I): 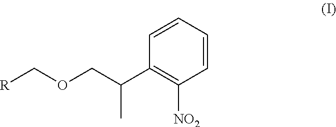
[wherein, in the formula (I), R represents a biomolecule].

23. The biomolecule according to claim 21 or 22, which is a base.

24. The biomolecule according to claim 23, wherein the base is thymine or guanine.

25. The biomolecule according to claim 24, which is represented by the following formula (II): 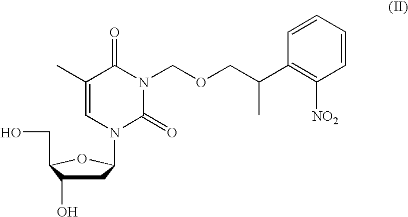
or the following formula (III): [chemical structure]


SEQUENCE LISTING (15 sequences)

SEQ ID NO: 1 - DNA, 24 bp, Artificial
  Synthetic DNA
agctgtccggcgtagaggatcgag

SEQ ID NO: 2 - DNA, 17 bp, Artificial
  Synthetic DNA
ctcgatcctctacgccg

SEQ ID NO: 3 - DNA, 24 bp, Artificial
  Synthetic DNA
aattcaccgtcaccctggatgctg

SEQ ID NO: 4 - DNA, 24 bp, Artificial
  Synthetic DNA
aattgtccggcgtagaggatcgag

SEQ ID NO: 5 - DNA, 36 bp, Artificial
  Synthetic DNA
atcatcagtaacccgtcaccgtcaccctggatgctg

SEQ ID NO: 6 - DNA, 36 bp, Artificial
  Synthetic DNA
acgggttactgatgatgtccggcgtagaggatcgag

SEQ ID NO: 7 - DNA, 4361 bp, Artificial
  Cloning vector pBR322
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 8 - DNA, 2501 bp, Artificial
  Cloning vector pBR322
tcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttc

SEQ ID NO: 9 - DNA, 5115 bp, Artificial
  Cloning vector pQBI T7-GFP
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatccggatatagttcctcctttcagcaaaaaacccctcaagacccgtttagaggccccaaggggttatgctagttattgctcagcggtggcagcagccaactcagcttcctttcgggctttgttagcagccggatcctcagttgtacagttcatccatgccatgtgtaatcccagcagctgttacaaactcaagaaggaccatgtggtctctcttttcgttgggatctttcgaaagggcagattgtgtggacaggtaatggttgtctggtaaaaggacagggccatcgccaattggagtattttgttgataatggtctgctagttgaacgcttccatcttcaatgttgtggcgggtcttgaagttcactttgattccattcttttgtttgtctgccatgatgtatacattgtgtgagttatagttgtattccaatttgtgtcccagaatgttgccatcttccttgaagtcaataccttttaactcgattctattaacaagggtatcaccttcaaacttgacttcagcacgtgtcttgtagttgccgtcatctttgaagaagatggtcctttcctgtacataaccttcgggcatggcactcttgaaaaagtcatgccgtttcatatgatccgggtatcttgaaaagcattgaacaccatagcacagagtagtgactagtgttggccatggaacaggcagtttgccagtagtgcagatgaacttcagggtaagttttccgtatgttgcatcaccttcaccctctccactgacagagaacttgtggccgttaacatcaccatctaattcaacaagaattgggacaactccagtgaagagttcttctcctttgctagccatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 10 - DNA, 1031 bp, Artificial
  Cloning vector pQBI T7-GFP
ctggatgctgtaggcataggcttggttatgccggtactgccgggcctcttgcgggatatccggatatagttcctcctttcagcaaaaaacccctcaagacccgtttagaggccccaaggggttatgctagttattgctcagcggtggcagcagccaactcagcttcctttcgggctttgttagcagccggatcctcagttgtacagttcatccatgccatgtgtaatcccagcagctgttacaaactcaagaaggaccatgtggtctctcttttcgttgggatctttcgaaagggcagattgtgtggacaggtaatggttgtctggtaaaaggacagggccatcgccaattggagtattttgttgataatggtctgctagttgaacgcttccatcttcaatgttgtggcgggtcttgaagttcactttgattccattcttttgtttgtctgccatgatgtatacattgtgtgagttatagttgtattccaatttgtgtcccagaatgttgccatcttccttgaagtcaataccttttaactcgattctattaacaagggtatcaccttcaaacttgacttcagcacgtgtcttgtagttgccgtcatctttgaagaagatggtcctttcctgtacataaccttcgggcatggcactcttgaaaaagtcatgccgtttcatatgatccgggtatcttgaaaagcattgaacaccatagcacagagtagtgactagtgttggccatggaacaggcagtttgccagtagtgcagatgaacttcagggtaagttttccgtatgttgcatcaccttcaccctctccactgacagagaacttgtggccgttaacatcaccatctaattcaacaagaattgggacaactccagtgaagagttcttctcctttgctagccatatgtatatctccttcttaaagttaaacaaaattatttctagaggggaattgttatccgctcacaattcccctatagtgagtcgtattaatttcgcgggatcgagatctcgatcctct

SEQ ID NO: 11 - DNA, 33 bp, Artificial
  Synthetic DNA
accgccacctccgttgtacagttcatccatgcc

SEQ ID NO: 12 - DNA, 34 bp, Artificial
  Synthetic DNA
aggatccggctgctaacaaagcccgaaaggaagc

SEQ ID NO: 13 - DNA, 34 bp, Artificial
  Synthetic DNA
agccggatcctcagttgtacagttcatccatgcc

SEQ ID NO: 14 - DNA, 36 bp, Artificial
  Synthetic DNA
aggtggcggtggcaaaggagaagaactcttcactgg

SEQ ID NO: 15 - DNA, 701 bp, Artificial
  Cloning vector pQBI 67 (BFP gene)
agttcatccatgccatgtgtaatcccagcagctgttacaaactcaagaaggaccatgtggtctctcttttcgttgggatctttcgaaagggcagattgtgtggacaggtaatggttgtctggtaaaaggacagggccatcgccaattggagtattttgttgataatggtctgctagttgaacgcttccatcttcaatgttgtggcggatcttgaagttcgctttgattccattcttttgtttgtctgccatgatgtatacattgtgtgagttatagttgtattccaatttgtgtccaagaatgtttccatcttctttaaaatcaataccttttaactcgattctattaacaagggtatcaccttcaaacttgacttcagcacgtgtcttgtagttgccgtcatctttgaagaagatggtcctttcctgtacataaccttcgggcatggcactcttgaaaaagtcatgccgtttcatatgatccgggtatcttgaaaagcattgaacaccatgagagagagtagtgactagtgttggccatggaacaggcagtttgccagtagtgcagatgaacttcagggtaagttttccgtatgttgcatcaccttcaccctctccactgacagagaacttgtggccgttaacatcaccatctaattcaacaagaattgggacaactccagtgaagagttcttctccttt